(19) United States
(12) Patent Application Publication
(10) Pub. No.: US 2022/0304313 A1
(43) Pub. Date: Sep. 29, 2022

(54) METHODS FOR DEGRADING AHL SIGNALING MOLECULES OR PREVENTING PLANT DISEASE CAUSED THEREBY

(71) Applicant: South China Agricultural University, Guangzhou City, Guangdong (CN)

(72) Inventors: Lianhui ZHANG, Guangzhou City (CN); Huishan WANG, Guangzhou City (CN); Lisheng LIAO, Guangzhou City (CN); Wenting WU, Guangzhou City (CN); Lingling DONG, Guangzhou City (CN); Xinghui FAN, Guangzhou City (CN)

(21) Appl. No.: 17/701,204

(22) Filed: Mar. 22, 2022

(30) Foreign Application Priority Data
Mar. 22, 2021 (CN) 202110303041.8
Mar. 22, 2021 (CN) 202110303045.6
Mar. 22, 2021 (CN) 202110304249.1

(51) Int. Cl. A01N 63/27; C12N 1/20

(52) U.S. Cl. CPC A01N 63/27; C12N 1/205

(57) ABSTRACT

The present disclosure belongs to the technical field of biological control and prevention, and it was found that Pseudomonasnitroreducens strain HS-18 has good degradation activity on AHLs with C4-C14 chain length, with an efficient and notable degradation effect, providing a new biocontrol agent for prevention and treatment of AHLs-mediated pathogens provides and broadening the quorum quenching species of AHL signaling molecules. By using molecular biology technologies, AHL-quenching genes for HS-18, i.e., the encoding genes aigA and aigC of N-acyl homoserine lactone acyl transferase are cloned. The genes aigA and aigC have broad-spectrum and efficient quenching activities on AHLs with different side chain lengths and different side chain substituents. The expression of aigA and aicC can significantly weaken the motility of AHL-mediated pathogens, the formation of biofilm and the production of virulence factors and significantly weaken the pathogenicity of pathogens to the host plant.

### CROSS REFERENCE TO RELATED APPLICATION

This application claims the priority to Chinese patent application No. 202110304249.1, titled “A gene aigC encoding N-acyl homoserine lactone acyl transferase and use thereof”, Chinese patent application No. 202110303041.8, titled “Use of Pseudomonas nitroreducens HS-18 in prevention and treatment of pathogens mediated by AHL” and Chinese patent application No. 202110303045.6, titled “A gene aigA encoding N-acyl homoserine lactone acyl transferase and use thereof”, all of which were filed on Mar. 22, 2021 and the entire contents of which are incorporated herein by reference.

### TECHNICAL FIELD

The present disclosure relates to the technical field of molecular biological control, in particular to a method for degrading AHL signaling molecules or for preventing and treating plant disease caused by AHL signaling molecules.

### BACKGROUND

The application of pesticides and antibiotics is the most common method for preventing pathogens. However, the long-term abuse of pesticides and antibiotics has caused threats to environmental safety and human and animal health, and even caused drug resistance to microorganisms. Therefore, a new and environmentally friendly antimicrobial method needs to be developed urgently.

Quorum sensing (QS) is a communication mechanism between cells, and quorum sensing signals are the “language” to achieve this communication mechanism. Quorum sensing signals involve small molecular compounds synthesized and secreted by bacteria intracellularly, which are produced throughout the entire cell growth cycle and can be freely diffused or actively transported to the extracellular environment. When the number of cells increases, the concentration of extracellular quorum sensing signals also increases, and cells sense the number of cells in the environment by sensing the concentration of quorum sensing signals. When the concentration of quorum sensing signals reaches a certain threshold, the signal molecules bind to receptor proteins and activate signal transduction to achieve the expression of target genes. Many pathogens use quorum sensing signals to regulate the expression of virulence factors and various physiological activities (for example, motility, formation of biofilm, exoenzyme production, plasmid transfer, drug resistance, bioluminescence, etc.) so that they have a better ability to adapt to the surrounding environment and has a stronger pathogenicity to the host plant.

Acyl homoserine lactone (N-acyl homoserine lactones, AHL)-based signals were first discovered more than 40 years ago in the marine bacteria Vibrio fischeri and Vibrio harvetii, which are the earliest and most thoroughly studied quorum sensing signals molecules among the Gram negative bacteria. AHLs are widely present in a variety of Gram negative bacteria, especially in a variety of pathogens, such as Pectobacterium carotovum, Pseudomonas aeruginosa, Burkholderia cepacia, Agrobacterium tumefaciens, Ralstonia solanacearum, Dickeya zeae, Erwiniacarotovora, Pantoea stewartii subsp. stewartii, etc. Most of the AHLs in these pathogens consist of a conserved N-acyl homoserine lactone ring and acyl chains with 4-18 carbon atoms and differ in side chain modifications.

Quorum quenching is an approach to block or destroy the quorum sensing system, which can be achieved through three main approaches: inhibiting the activity of quorum sensing signals synthase, inhibiting the activity of quorum sensing signals receptor protein, and using quenching enzymes to modify or enzymatically hydrolysize quorum sensing signals molecules. The discovery and application of quorum quenching genes or quenching enzymes has become one of the current research hotspots of quorum quenching. The earliest identified AHL-quenching enzyme is AHL lactonase—AiiA found in Bacillus thuringiensis. AHL lactonase can break up the lactone bond of the lactone ring in the AHLs molecule, thereby inactivating the AHL signal molecule. The expression of AiiA in pathogens can significantly reduce the yield of the virulence factor of the pathogens and the virulence to the host plant, and the expression of AiiA in the plant can effectively improve the disease resistance of the host plant to the pathogens. Upon the identification of AHL lactonase, AHL acyl transferase, which can cleave the amide bond between the AHL lactone ring and the acyl chain, and AHL oxidoreductase, which acts on the hydrogen atom of the side chain, were also discovered successively.

It was found in the study that the expression of most of the identified AHL-quenching genes in pathogens can weaken the virulence of pathogens, and the expression of AHL-quenching genes in host plant can improve the host's resistance to pathogens, AHL-quenching genes or the encoded product of AHL-quenching genes, AHL-quenching enzymes, have been widely and effectively applied in biocontrol in agriculture, aquaculture, and biofilm reactors for sewage treatment. Therefore,—identification and application of AHL-quenching genes will lay a foundation for enriching the resources of AHLs quorum quenching preparations, and is of great practical application value to the biological control of quorum quenching pathway.

### SUMMARY

In order to solve the above problems in the prior art, the present disclosure, in a first aspect, provides a method for degrading AHL signaling molecules or preventing and treating plant disease caused by AHL signaling molecules.

The present disclosure provides a method for degrading an AHL signaling molecules using pseudomonas nitroreducens HS-18, wherein the AHL signaling molecule is selected from the group consisting of C4-HSL, C6-SHL, 3—O—C6-HSL, C8-HSL, 3—OH—C8-HSL, C10-HSL, 3—OH—C10—HSL, C12-HSL, 3—O—C12-HSL, 3—OH—C12-HSL, and 3—OH—C14-HSL.

In one preferred embodiment of the present disclosure, pH and temperature for degrading the AHL signaling molecule using Pseudomonas nitroreducens HS-18 are 6.8-7.2 and 28-37° C., respectively.

The present disclosure also provides a method for preventing and treating AHL signaling molecule-dependent pathogens by using Pseudomonas nitroreducens HS-18, comprising a step of co-inoculating a liquid bacterial culture containing Pseudomonas nitroreducens HS-18 and the AHL signaling molecule-dependent pathogens in a plant.

In one preferred embodiment of the present disclosure, co-inoculating is conducted by the steps of mixing a liquid bacterial culture containing Pseudomonas nitroreducens HS-18 with the AHL signaling molecule-dependent pathogens, and applying to diseased part of the plant.

In one preferred embodiment of the present disclosure, the AHL signaling molecule-dependent pathogen is selected from the grouping consisting of Pectobacterium carotovorum subsp. carotovorum, Burkholderia cepacia, Pseudomonas aeruginosa, Ralstonia solanacearum, Agrobacterium tumefaciens, Dickeya zeae, Erwinia or Pantoea stewartii subsp. stewartii.

In one preferred embodiment of the present disclosure, the AHL signaling molecule-dependent pathogen is selected from the group consisting of 3—O—C6-HSL-dependent pathogenic Pectobacterium carotovorum subsp. carotovorum, C8-HSL-dependent pathogenic Burkholderia cepacia, C4-HSL-dependent and 3—OH—C12-HSL-dependent pathogenic Pseudomonas aeruginosa, C6-HSL-dependent and C8-HSL-dependent pathogenic Ralstonia solanacearum, 3—O—C8-HSL-dependent pathogenic Agrobacterium tumefaciens, 3—O—C6-HSL-dependent pathogenic Dickeya zeae, 3—O—C6-HSL-dependent pathogenic Erwinia.

In one preferred embodiment of the present disclosure, the preventing and treating the AHL signaling molecule-dependent pathogens include:

(1) reducing production of the AHLs; and/or,
(2) reducing motility of the pathogen; and/or,
(3) reducing formation of biofilm of the pathogen; and/or,
(4) reducing production of protease of the pathogen; and/or,
(5) weakening pathogenicity of the pathogen.

The present disclosure also provides a gene encoding N-acyl homoserine lactone acyl transferase derived from the Pseudomonas nitroreducens HS-18, comprising a gene aigA or a gene of aigC; wherein the nucleotide sequence of the gene aigA is set forth in SEQ ID NO: 1, and the nucleotide sequence of the gene aigC is set forth in SEQ ID NO: 2.

The present disclosure also provides a recombinant vector comprising the gene encoding N-acyl homoserine lactone acyl transferase as described above, which is inserted into the recombinant vector.

In one preferred embodiment of the present disclosure, the recombinant vector is a recombinant vector obtained by inserting the gene aigA or the gene aigC into the broad-host vector pBBR1 for heterologous expression of the gene encoding N-acyl homoserine lactone acyl transferase; or

the recombinant vector is a recombinant vector for protein prokaryotic expression of N-acyl homoserine lactone acyl transferase and is obtained by inserting the gene aigA or the gene aigC into a protein prokaryotic expression vector pET32a.

The present disclosure also provides a recombinant bacterium comprising the recombinant vector described in the above technical solution.

In one preferred embodiment of the present disclosure, original bacterium of the recombinant vector is a non-pathogen or an AHL signaling molecule-dependent pathogen.

The present disclosure also provides a formulation for preventing and treating AHL signaling molecule-dependent pathogen or for degrading a AHL signaling molecule, wherein the formulation comprises the recombinant bacterium of the above technical solution, or comprises N-acyl homoserine lactone acyl transferase; and the amino acid sequence of N-acyl homoserine lactone acyl transferase is set forth in SEQ ID NO: 3 or SEQ ID NO: 4.

Compared with the prior arts, the present disclosure has the following beneficial effects.

In the study of the present disclosure, it was found that Pseudomonas nitroreducens strain HS-18 has good degradation activity to AHLs with C4-C14 chain length for detection, and the degradation effect is highly efficient and significant, which provides a new biocontrol agent for biological control of pathogens mediated by AHLs. On the basis of the fact that Pseudomonas nitroreducens HS-18 can efficiently degrade AHL signaling molecules with C4-C14 acyl chain length, N-acyl homoserine lactone acyl transferase encoding genes aigA and aigC were cloned through molecular biology technology in the present disclosure. The gene aigA or aigC has broad-spectrum and efficient quenching activity on AHLs with different side chain length and different side chain substituents, and has good degradation effect on N-acyl homoserine lactone modified with different carbon chain length and different substituents. The expression of the N-homoserine lactone quenching gene in the AHL signaling molecule-dependent pathogens can significantly attenuate the motility of the pathogens, formation of biofilms and production of virulence factors such as exoenzymes, meanwhile reduces the pathogenicity of the host plant. The present disclosure lays an important foundation for enriching the encoding gene resources of the N-acyl homoserine lactone acyl transferase.

### BRIEF DESCRIPTION OF THE DRAWINGS

FIG. 1 shows the detection of degradation activity of Pseudomonas nitroreducens HS-18 on AHLs with different acyl chain lengths (C4-C14).

FIG. 2 is a graph showing the biological control effect of Pseudomonas nitroreducens HS-18 on 3—O—C6-HSL (OHHL)-dependent pathogenic Pectobacterium carotovorum subsp. carotovorum on the host plant of potatoes.

FIG. 3 is a graph showing the biological control effect of Pseudomonas nitroreducens HS-18 on C8-HSL (OHL)-dependent pathogenic Burkholderia cepacia on the host plant of onions.

FIG. 4 is a graph showing the biological control effect of Pseudomonas nitroreducens HS-18 on C4-HSL (BHL) and 3—O—C12-HSL (OdDHL)-dependent pathogenic Pseudomonas aeruginosa on the host plant of lettuce.

FIG. 5A and FIG. 5B are graphs showing the recombinant Escherichia coli DH5α (aigC) (FIG. 5B) expressing the gene encoding N-acyl homoserine lactone acyl transferase in broad-host-range vector pBBR1 according to the present disclosure and effect of Escherichia coli DH5α containing an empty vector (pBBR1) (FIG. 5A) after culturing for 36 h to degrade AHLs.

FIG. 6 shows the phylogenetic analysis of the N-acyl homoserine lactone acyl transferase protein AigC of the present disclosure.

FIG. 7 shows the expression of AigC protein; M: Protein Marker; 1: Expression of total proteins in recombinant bacteria expressing AigC protein; 2: Expression of total proteins in BL21 (DE3) containing empty vector pET32a; 3: Expression of intracellular protein in recombinant bacteria expressing AigC protein; 4: recombinant bacterium expression in BL21 (DE3) containing empty vector pET32a.

FIG. 8A-FIG. 8E show the effect of expression of the gene aigC encoding N-acyl homoserine lactone acyl transferase of the present disclosure in AHL-dependent pathogen Burkholderia cepacia H111 on the growth of H111, self-produced AHL yield and virulence factors. FIG. 8A shows the growth curve of Burkholderia cepacia, FIG. 8B shows the production of intracellular C8-HSL, FIG. 8C shows the motility of Burkholderia cepacia, FIG. 8D shows Burkholderia cepacia formation of biofilm, and

FIG. 8E shows the yield of Burkholderia cepacia protease.

FIG. 9 shows the effect of expression of the aigC encoding N-acyl homoserine lactone acyl transferase of the present disclosure in AHL-dependent pathogen Burkholderia cepacia H111 on the pathogenicity of H111.

FIG. 10A-FIG. 10E show the effect of expression of the gene aigC encoding N-acyl homoserine lactone acyl transferase of the present disclosure in AHL-dependent pathogen Pseudomonas aeruginosa PAO1 on the growth of PAO1, self-produced AHL production and virulence factors. FIG. 10A shows the growth curve of Pseudomonas aeruginosa; FIG. 10B shows yield of intracellular C4-HSL; FIG. 10C shows the intracellular production of 3—O—C12-HSL in Pseudomonas aeruginosa; FIG. 10D shows the motility of the Pseudomonas aeruginosa; and FIG. 10E shows the production of Pseudomonas aeruginosa protease.

FIG. 11A and FIG. 11B show the effect of the expression of the gene aigC encoding N-acyl homoserine lactone acyl transferase of the present disclosure in AHL-dependent pathogen Pseudomonas aeruginosa PAO1 on the pathogenicity of PAO1. FIG. 11A shows the effect on the pathogenicity of lettuce, and FIG. 11B shows the effect on the pathogenicity of cabbage.

FIG. 12A and FIG. 12B are graphs showing the recombinant Escherichia coli DH5α (aigA) (FIG. 12B) expressing the gene encoding N-acyl homoserine lactone acyl transferase in broad-host-range vector pBBR1 according to the present disclosure and the effect of Escherichia coli containing an empty vector DH5α (pBBR1) (FIG. 12A) after culturing for 36 h to degrade AHLs.

FIG. 13 is graph showing the evolution tree of N-acyl homoserine lactone acyl transferase protein AigA of the present disclosure.

FIG. 14 shows the expression of AigC protein; M: Protein Marker; 1: Expression of total proteins in recombinant bacteria expressing AigC protein; 2: Expression of total proteins in BL21 (DE3) containing empty vector pET32a; 3: Expression of intracellular protein in recombinant bacteria expressing AigC protein; 4: recombinant bacterium expression in BL21 (DE3) containing empty vector pET32a.

FIG. 15A-FIG. 15E show the effect of expression of the gene aigC encoding N-acyl homoserine lactone acyl transferase of the present disclosure in AHL-dependent pathogen Burkholderia cepacia H111 on the growth of H111, self-produced AHL yield and virulence factors. FIG. 15A shows the growth curve of Burkholderia cepacia; FIG. 15B shows yield of intracellular C8-HSL; FIG. 15C shows the motility of Burkholderia cepacia; FIG. 15D shows Burkholderia cepacia formation of biofilm; and FIG. 15E shows the yield of Burkholderia cepacia protease.

FIG. 16 shows the effect of expression of the aigC encoding N-acyl homoserine lactone acyl transferase of the present disclosure in AHL-dependent pathogen Burkholderia cepacia H111 on the pathogenicity of H111.

FIG. 17A-FIG. 17E show the effect of expression of the gene aigC encoding N-acyl homoserine lactone acyl transferase of the present disclosure in AHL-dependent pathogen Pseudomonas aeruginosa PAO1 on the growth of PAO1, self-produced AHL yield and virulence factors. FIG. 17A shows the growth curve of Pseudomonas aeruginosa; FIG. 17B shows the intracellular production of C4-HSL in Pseudomonas aeruginosa; FIG. 17C shows the intracellular production of 3—O—C12-HSL in Pseudomonas aeruginosa; FIG. 17D shows the motility of Pseudomonas aeruginosa; and FIG. 17E shows the production of Pseudomonas aeruginosa protease.

FIG. 18A and FIG. 18B show the effect of the expression of the gene aigC encoding N-acyl homoserine lactone acyl transferase of the present disclosure in AHL-dependent pathogen Pseudomonas aeruginosa PAO1 on the pathogenicity of PAO1. FIG. 18A shows the effect on the pathogenicity of lettuce, and FIG. 18B shows the effect on the pathogenicity of cabbage.

### DETAILED DESCRIPTION OF THE EMBODIMENTS

### Biological Deposit Information

Pseudomonas nitroreduction strain, designated as HS-18, was deposited in China Center for Type Culture Collection (CCTCC) on May 12, 2017. The deposit number is CCTCC NO: M2017257, and the deposit address is: Wuhan University, Wuhan City, China.

Firstly, the present disclosure provides the use of Pseudomonas nitroreduction HS-18 in degrading an AHL signaling molecule, wherein the AHL signaling molecule is selected from the group consisting of C4-HSL, C6-SHL, 3—O—C6-HSL, C8-HSL, 3—OH—C8-HSL, C10-HSL, 3—OH—C10-HSL, C12-HSL, 3—O—C12-HSL, 3—OH—C12-HSL, and 3—OH—C14-HSL.

Although the structural AHLs mainly different in the length of side chains and the types of substituents, strains capable of degrading short-chain AHLs may not necessarily degrade medium- and long-chain AHLs, and vice versa. There also are broad-spectrum AHL-quenching bacteria that can degrade AHLs with different chain lengths. For example, 7 AHL-quenching bacteria species were found in previous studies, RO1 and RO5 from the Stenotrophomonas genus, R02 from the Bacillus genus and RO7 from the Pseudomonas genus can only degrade short-chain and medium-chain AHLs (C4-C10), but cannot degrade long-chain AHLs (C12-C14), while RO3 and RO4 from the Bacillus genus and RO6 from the Delftia genus can degrade short-chain and medium-chain AHLs, and it can also degrade long-chain C14-HSL, but it cannot degrade 12-carbon chain-length AHLs having a chain length of 12 carbons. It can be seen that the degradation ability of different types of AHL signaling molecules by strains of the same species may be different, and there are also different strains that can degrade the same type of AHL signaling molecules at the same time, which may be based on AHL different types of quenching enzymes in different quenching bacteria, and on the specificity of different for degraded substrates. In the case of specific strains to degrade AHL signaling molecules, specific research is needed to be done for more information. Through research, it is found in the present disclosure that Pseudomonas nitroreduction HS-18 can efficiently degrade AHL signaling molecules with an acyl chain length of C4-C14, thus broadening the species of quorum quenching bacteria of AHL signaling molecules.

The present disclosure also provides the use of Pseudomonas nitroreduction HS-18 in preventing and treating a plant disease mediated by an AHL signaling molecule, wherein the AHL signaling molecule is selected from the group consisting of C4-HSL, C6-SHL, 3—O—C6-HSL, C8-HSL, 3—OH—C8-HSL, C10-HSL, 3—OH—C10-HSL, C12-HSL, 3—O—C12-HSL, 3—OH—C12-HSL, and 3—OH—C14-HSL.

In the present disclosure, the above use comprises co-inoculating plants with the Pseudomonas nitroreduction HS-18 and the AHL signaling molecule-dependent pathogens.

In the present disclosure, the use comprises mixing the above-mentioned liquid bacterial culture of pseudomonas nitroreducens HS-18 with the AHL signaling molecule-dependent pathogens and dotting the liquid bacterial culture onto the diseased part of the plant; wherein PBS buffer is used to prepare the liquid bacterial culture of Pseudomonas nitroreduction HS-18. The concentration of bacterial suspension is not strictly limited, and the concentration can be adjusted based on the actual severity of plant disease and effect of use.

In the present disclosure, the AHL signaling molecule-dependent pathogen is selected from the group consisting of 3—O—C6-HSL-dependent pathogenic Pectobacterium carotovorum subsp. carotovorum, C8-HSL-dependent pathogenic Burkholderia cepacia, C4-HSL-dependent and 3—OH—C12-HSL-dependent pathogenic Pseudomonas aeruginosa, C6-HSL-dependent and C8-HSL-dependent pathogenic Ralstonia solanacearum, 3—O—C8-HSL-dependent pathogenic Agrobacterium tumefaciens, 3—O—C6-HSL-dependent pathogenic Dickeya zeae, 3—O—C6-HSL-dependent pathogenic Erwinia.

In the present disclosure, pH and temperature for degrading the AHL signaling molecule using Pseudomonas nitroreduction HS-18 are 6.8-7.2 and 28-37° C., respectively.

In the present disclosure, the culture medium for the Pseudomonas nitroreduction HS-18 is LB medium, and the formula is: trypton 10.0 g/L, yeast extract 5.0 g/L, NaCl 10.0 g/L, and pH 6.8-7.2.

The present disclosure also provides the use of Pseudomonas nitroreduction HS-18 in preparation of a biological agent for resisting AHL signaling molecule-mediated pathogenic plant diseases, wherein the AHL signaling molecule is selected from the group consisting of C4-HSL, C6-SHL, 3—O—C6-HSL, C8-HSL, 3—OH—C8-HSL, C10-HSL, 3—OH—C10-HSL, C12-HSL, 3—O—C12-HSL, 3—OH—C12-HSL, and 3—OH—C14-HSL.

The present disclosure also provides the use of Pseudomonas nitroreduction HS-18 in preparation of a biological agent for resisting AHL signaling molecule-mediated pathogenic plant diseases, wherein the AHL signaling molecule is selected from the group consisting of C4-HSL, C6-SHL, 3—O—C6-HSL, C8-HSL, 3—OH—C8-HSL, C10-HSL, 3—OH—C10-HSL, C12-HSL, 3—O—C12-HSL, 3—OH—C12-HSL, and 3—OH—C14-HSL.

It was found in the early study of the present disclosure that on the basis of the fact that Pseudomonas nitroreduction HS-18 can efficiently degrade AHL signaling molecules with an acyl chain length of C4-C14, genes aigC and aigA encoding N-acyl homoserine lactone acyl transferase was cloned by molecular biology technology, and the nucleotide sequence of aigC is set forth in SEQ ID NO: 2, specifically:

| atgaaacgca ctttgactgt cctcgctgtc gttgtcgtcg ctgccgcggc gggcgctggc | 60 |
|---|---|
| tggtacctgc acggcaagca gccggtacgc gacggacaac tgccgctggc cggcctcgcc | 120 |
| agcgaagtca cggttcgcta cgacgagcgt ggcgtgccgc acatcaaggc cggcagcgag | 180 |
| gaggacatgt accgggcgat cggttacgtg catgcccagg accggctgtt ccagatggaa | 240 |
| atactgcggc gcctgtcccg cggcgagctg gccgaagtgc tcgggcccaa gctggtggac | 300 |
| accgaccgca tgttccgtag cctgcgcatc cgcgaccacg cggcggagta tgtgaagacg | 360 |
| caggacaaga attccccggc ctggaaggcc ctggtcgcct acctcgatgg cgtcaaccag | 420 |
| ttccaggaca gccacccgcg cccggtggag ttcgacatcc tcggcatacc caagcgcccc | 480 |
| ttcacgcccg aggacaccgt cagcgtcgcc ggctacatgg cctacagctt cgccgccgcc | 540 |
| ttccgtaccg aaccggtgct gacctacgtg cgcgaccaac tgggcgcgga ttacctgaaa | 600 |
| gtcttcgacc tcgactggca ccccaacggc gtcctcacgc cgtccccgtt ggccgccgcc | 660 |
| gactggcagg acatgagcgc catcgcccag ctcagccacg cggccctgga gaaggccggc | 720 |
| ctgccgcagt tcgaaggcag caacgcctgg gccgtgtccg gcagccgcac caagagcggc | 780 |
| aagccgctgc tggcgggtga cccacacatc cgcttcgccg tgccggcagt gtggtacgag | 840 |
| atgcaggcca gcgcgccggg gttcgagctg tacggccact accaggcgct caacccgttc | 900 |
| gcctcgctgg gccataacct gcagttcggc tggagcctga ccatgttcca gaacgacgac | 960 |
| gtcgacctgg tcgccgagaa ggtcaacccg gacaacccca accaggtctg gtaccacggc | 1020 |
| cagtgggtgg acctgaagag cgaggagcag agcatcgccg tgaaaggcga ggcgccagtg | 1080 |
| aagatcaccc tgcgcagctc gccccacggt ccgctggtca atgacgcgct gggcactgcc | 1140 |
| gcgggcaaga caccggtggc catgtggtgg gccttcctgg aaacccagaa cccgatcctc | 1200 |
| gatgccttct acgagctgaa ccgcgccgat acccttgcca aggcccgcac ggcggcttcg | 1260 |
| aagatccagt cgccgggcct caacgtggta tgggccaacg cccgcggcga catcggctgg | 1320 |
| tgggcatcgg cgcaattgcc ggtacgcccg gacggggtca acccgaactt cctgctcgac | 1380 |
| ggcgccagcg gccaggccga caagaccggc ttctacccct tcagcgagaa cccgcaggaa | 1440 |
| gaaaacccgg cgcgcggcta catcgtctcg gccaacttcc agccggtacc ggccaacggt | 1500 |
| cgcccggtgc cgggctacta caacctgccc gaccgcggtc agcaactgaa caagcgcctg | 1560 |
| tccgacgatt cggtgaaatg ggacctgcag aacagccagg cgctgcaact ggacaccgcc | 1620 |
| accggctatg gcccgcgctt cctcaagccg ctgctgccga tcctgcgtga agccgcggca | 1680 |
| accgacgaag agaaggcgct ggtggagagc ctggccaact ggcagggcga ccatccgctg | 1740 |
| gactccgtga ccgccacgct gttcaaccag ctcctctacc aggtggcaga cggcgccatg | 1800 |
| cgcgacgaga tgggcgacgc cttcttcgac aacctgctgt ccacccgcgt gctcgacgtg | 1860 |
| gccctgccgc gcctggccgc cgacgagggc tcgccctggt gggacaaccg caagacgccg | 1920 |
| cagacaa ctcgcgcgga tatcgtcaag gccgcctgga aaggcagcct ggcgcatctg | 1980 |
| cgcaccaccc tcggccagga ctcgaaacag tggctgtggg gtaaggcgca caccctgacc | 2040 |
| cacggccacc cgctgggcca gcagaagccg ctggatcgcc tcttcaatgt cggcccattc | 2100 |
| gccgcgccgg gcggccacga ggtaccgaac aacctctccg cccgcgtcgg cccggcaccc | 2160 |
| tggcaggtgg tctacggccc gtccacccgt cgcctgatcg acttcgccga cccggcccac | 2220 |
| agcctgggca tcaacccggt gggccagagc ggcgtgccct tcgacaagca ctacgacgac | 2280 |
| caggccgaag cctacatcga aggccagtac ctgccgcagc actacgatga aaacgaggtg | 2340 |
| aaggccaaca ccaagggcat cctgcggctg atccccgtcc gtcgctga | 2388; |
| The nucleotide sequence of aigA is set forth in SEQ ID NO: 1, specifically: | |
| atggccccgc gtgccttccg ccggcttccc agcttcggcc ttgccgccac cgtcactgcc | 60 |
| gctgtcagcc ttaccggctg ccagggctgg ctgaatgacc gctattccga cagcctgccg | 120 |
| ccgagctacg gcgtgcagcc gatcaagggc ctggccgaca acgtgtcgat ccgccgcaac | 180 |
| agcctgggca tgccgctgat cgagaccagc accttccacg acgcgctgtt caccctcggt | 240 |
| tatgtgcacg ccagcgaccg catcagccag atggtcggcc tgcgcctgat ggcccagggt | 300 |
| cgcctgtcgg aaatggtcgg ccccggcgcc ctggaaaccg accgattcat gcgcacggtg | 360 |
| aacctgaaga aggccgcgga cgccctctac gccggcgcct caccgcgcct caagcgtttc | 420 |
| ttcgagacct acgcgcgcgg cgtcaacgcc tacctgtacc gctaccgcga caagctgccg | 480 |
| atggacctcg ccgagtccgg ctaccgcccc gagtactgga agccggaaga ttccgcactg | 540 |
| gtcttcagcc tgctgaactt cggcctggcg gtgaacctgc aggaggaaat cgcctccctg | 600 |
| gtactggcgc agaaagtcgg tgccgacaag ctgccctggc tgacgccgat ctatccggac | 660 |
| gaagcgctgc cgttcgagga agcggacaaa ctcaagggcc tgcgcctgga cggcaggatc | 720 |
| gccggcctcg acgccctgga tcgtgcggca ggccaggtcg ccgccctgca gatgatggggc | 780 |
| gtcgccgcct ccaacaactg ggccatcgcc ccgcagcgca cgcgcagcgg caagagcatc | 840 |
| ctggccaacg acacccacct gccgctgtcc atgccctcgg tgtggaacta cgtgcagatc | 900 |
| cgctcgccga aatacaacgc cgcgggcgtc tccatcgccg gcgtaccggg cgtggtcgcc | 960 |
| ggtttcaacg gcaagctggc ctggggcatg accatggtca tgggcgacaa ccaggacctt | 1020 |
| ttccttgagc aggtgaagcc ccagggcggc aagctctact acctcgccga tggccagtgg | 1080 |
| aaaccggcca tcgagcgcaa tgaaaccttc ttcatcaagg gccagagccc ggtccgcgaa | 1140 |
| gtcatctacg agactcgcca cggcccgctg ctcaacagcg cgctgggcga gcgcaagcac | 1200 |
| gacctgcaac cgctgccgct gtccagcggc tacggcatcg cctaccagag catccagggc | 1260 |
| gagaccgacc gcaccctcga cgccttcttc gacctgtccc gcgcgaagaa cgtcgagcag | 1320 |
| gccttcgacg ccacccgcga cgtgcgcgcc atggcgctga acatcgtgtt cgccgacgag | 1380 |
| aagcacatcg gctggcaggt caccgggcgc ttccccaacc gcaaggaagg ccgcggcctg | 1440 |
| ctgccttcgc ccggctggga cggccgctat gactgggacg gctacgccga tccgatcctc | 1500 |
| cacccgtccg accaggaccc gcagcagggc tggctgggca ccgccaacca ccgcagcgtg | 1560 |
| cagccaggct acggcgcgca gctgtccagt tcctggtact acccggagcg ctacgagcgc | 1620 |
| atcgcccaac tcgccggcgg cagcaagagc cacgactacc gcagcatgat cgccatgcag | 1680 |
| tacgaccaga ccacgccctt tgccggcaag ctgcaggcca tgttcgatgc accgggcatg | 1740 |
| gcgcagccgc tgaagaaagc catcgacgcc ctgcccgccg accagcgcgc ccgcgcccgc | 1800 |
| gaagcgctgg accggatcat ggcgttcgac ggcaagctct cggcgacctc cggcgacgcc | 1860 |
| gccctgtacg aagccttcct gcaggagagc agcaagcaga tcttcctcga tgaactgggc | 1920 |
| ccggaggaca gcccgtcctg gaaagccttc gtcgagaccg ccaacctctc ctactcggcc | 1980 |
| caggccgacc acctgctggg ccgtgacgac agcccgttct gggatgacac ccgcaccccg | 2040 |
| cagaaggaag acaaaccgac catcctcgcc cgcagcctgg ccgccgccac cgccttctgc | 2100 |
| gaacagaagc tgggcagcga ccgccgtacc tggcagtggg gcaagctgca cacctacgag | 2160 |
| tgggtcagcg acagcaccaa gatggcgccc aacctcagtg ccggcgagcg cgccagcctc | 2220 |
| ggcgcgatca agggctacct ggaccgcggc ccctacccgg ccggcggcga ccacagcacg | 2280 |
| ctgaacgtct cggcctacca ctggggccag gacttcaaca cctggctgat cccggccatg | 2340 |
| cgcatcgtcg tcgacttcgg ccagagcgaa ccgatgatcg gccttaacag cagtggccag | 2400 |
| tccggcaacc cggccagccc gcactacgca gacggcatcg acgcctggct gaagggcaac | 2460 |
| tacatgagct tccccttcca gtcgcagaac cttgagaagg tctacggcgt gaagcgcctg | 2520 |
| acgctggtgc cggagaagtg a | 2541. |

It can be understood that the above-mentioned recombinant vector containing the gene encoding N-acyl homoserine lactone acyl transferase also falls within the protection scope of the present disclosure, and the recombinant bacterium containing the recombinant vector also falls within the protection scope of the present disclosure.

In one preferred embodiment of the present disclosure, the recombinant vector is a recombinant vector for heterologous expression of a gene encoding N-acyl homoserine lactone acyl transferase, which is obtained by inserting the gene encoding N-acyl homoserine lactone acyl transferase into the broad-host-range vector pBBR1.

In one preferred embodiment of the present disclosure, the recombinant vector is obtained by inserting the gene encoding N-acyl homoserine lactone acyl transferase into a protein prokaryotic expression vector pET32a to produce a recombinant vector for protein prokaryotic expression of N-acyl homoserine lactone acyl transferase.

In one preferred embodiment of the present disclosure, the recombinant vector is obtained by introducing a recombinant vector that expresses the gene encoding N-acyl homoserine lactone acyl transferase in the broad-host-range vector pBBR1 into Escherichia coli DH5a.

In one preferred embodiment of the present disclosure, the recombinant vector is obtained by introducing a recombinant vector that expresses the gene encoding N-acyl homoserine lactone acyl transferase in the broad-host-range vector pBBR1 into AHL-dependent pathogens.

In yet another preferred embodiment of the present disclosure, the recombinant bacterium is obtained by introducing a recombinant vector that expresses the gene encoding N-acyl homoserine lactone acyl transferase in the protein prokaryotic expression vector pET32a into Escherichia coli BL21 (DE3).

The present disclosure also provides two kinds of N-acyl homoserine lactone acyl transferase, named AigC and AigA. The amino acid sequence of AigC is set forth in SEQ ID NO: 3, specifically:

Met Lys Arg Thr Leu Thr Val Leu Ala Val Val Val
Val Val Ala Ala Ala Ala Gly Ala Gly Trp Tyr Leu
His Gly Lys Gln Pro Val Arg Asp Gly Gln Leu Pro
Leu Ala Gly Leu Ala Ser Glu Val Thr Val Arg Tyr
Asp Glu Arg Gly Val Pro His Ile Lys Ala Gly Ser
Glu Glu Asp Met Tyr Arg Ala Ile Gly Tyr Val His
Ala Gln Asp Arg Leu Phe Gln Met Glu Ile Leu Arg
Arg Leu Ser Arg Gly Glu Leu Ala Glu Val Leu Gly
Pro Lys Leu Val Asp Thr Asp Arg Met Phe Arg Ser
Leu Arg Ile Arg Asp His Ala Ala Glu Tyr Val Lys
Thr Gln Asp Lys Asn Ser Pro Ala Trp Lys Ala Leu
Val Ala Tyr Leu Asp Gly Val Asn Gln Phe Gln Asp
Ser His Pro Arg Pro Val Glu Phe Asp Ile Leu Gly
Ile Pro Lys Arg Pro Phe Thr Pro Glu Asp Thr Val
Ser Val Ala Gly Tyr Met Ala Tyr Ser Phe Ala Ala
Ala Phe Arg Thr Glu Pro Val Leu Thr Tyr Val Arg
Asp Gln Leu Gly Ala Asp Tyr Leu Lys Val Phe Asp
Leu Asp Trp His Pro Asn Gly Val Leu Thr Pro Ser
Pro Leu Ala Ala Ala Asp Trp Gln Asp Met Ser Ala
Ile Ala Gln Leu Ser His Ala Ala Leu Glu Lys Ala
Gly Leu Pro Gln Phe Glu Gly Ser Asn Ala Trp Ala
Val Ser Gly Ser Arg Thr Lys Ser Gly Lys Pro Leu
Leu Ala Gly Asp Pro His Ile Arg Phe Ala Val Pro
Ala Val Trp Tyr Glu Met Gln Ala Ser Ala Pro Gly
Phe Glu Leu Tyr Gly His Tyr Gln Ala Leu Asn Pro
Phe Ala Ser Leu Gly His Asn Leu Gln Phe Gly Trp
Ser Leu Thr Met Phe Gln Asn Asp Asp Val Asp Leu
Val Ala Glu Lys Val Asn Pro Asp Asn Pro Asn Gln
Val Trp Tyr His Gly Gln Trp Val Asp Leu Lys Ser
Glu Glu Gln Ser Ile Ala Val Lys Gly Glu Ala Pro
Val Lys Ile Thr Leu Arg Ser Ser Pro His Gly Pro
Leu Val Asn Asp Ala Leu Gly Thr Ala Ala Gly Lys
Thr Pro Val Ala Met Trp Trp Ala Phe Leu Glu Thr
Gln Asn Pro Ile Leu Asp Ala Phe Tyr Glu Leu Asn
Arg Ala Asp Thr Leu Ala Lys Ala Arg Thr Ala Ala
Ser Lys Ile Gln Ser Pro Gly Leu Asn Val Val Trp
Ala Asn Ala Arg Gly Asp Ile Gly Trp Trp Ala Ser
Ala Gln Leu Pro Val Arg Pro Asp Gly Val Asn Pro
Asn Phe Leu Leu Asp Gly Ala Ser Gly Gln Ala Asp
Lys Thr Gly Phe Tyr Pro Phe Ser Glu Asn Pro Gln
Glu Glu Asn Pro Ala Arg Gly Tyr Ile Val Ser Ala
Asn Phe Gln Pro Val Pro Ala Asn Gly Arg Pro Val
Pro Gly Tyr Tyr Asn Leu Pro Asp Arg Gly Gln Gln
Leu Asn Lys Arg Leu Ser Asp Asp Ser Val Lys Trp
Asp Leu Gln Asn Ser Gln Ala Leu Gln Leu Asp Thr
Ala Thr Gly Tyr Gly Pro Arg Phe Leu Lys Pro Leu
Leu Pro Ile Leu Arg Glu Ala Ala Ala Thr Asp Glu
Glu Lys Ala Leu Val Glu Ser Leu Ala Asn Trp Gln
Gly Asp His Pro Leu Asp Ser Val Thr Ala Thr Leu
Phe Asn Gln Leu Leu Tyr Gln Val Ala Asp Gly Ala
Met Arg Asp Glu Met Gly Asp Ala Phe Phe Asp Asn
Leu Leu Ser Thr Arg Val Leu Asp Val Ala Leu Pro
Arg Leu Ala Ala Asp Glu Gly Ser Pro Trp Trp Asp
Asn Arg Lys Thr Pro Gln Lys Glu Thr Arg Ala Asp
Ile Val Lys Ala Ala Trp Lys Gly Ser Leu Ala His
Leu Arg Thr Thr Leu Gly Gln Asp Ser Lys Gln Trp
Leu Trp Gly Lys Ala His Thr Leu Thr His Gly His
Pro Leu Gly Gln Gln Lys Pro Leu Asp Arg Leu Phe
Asn Val Gly Pro Phe Ala Ala Pro Gly Gly His Glu
Val Pro Asn Asn Leu Ser Ala Arg Val Gly Pro Ala
Pro Trp Gln Val Val Tyr Gly Pro Ser Thr Arg Arg
Leu Ile Asp Phe Ala Asp Pro Ala His Ser Leu Gly
Ile Asn Pro Val Gly Gln Ser Gly Val Pro Phe Asp
Lys His Tyr Asp Asp Gln Ala Glu Ala Tyr Ile Glu
Gly Gln Tyr Leu Pro Gln His Tyr Asp Glu Asn Glu
Val Lys Ala Asn Thr Lys Gly Ile Leu Arg Leu Ile
Pro Val Arg Arg.

Met Ala Pro Arg Ala Phe Arg Arg Leu Pro Ser Phe
Gly Leu Ala Ala Thr Val Thr Ala Ala Val Ser Leu
Thr Gly Cys Gln Gly Trp Leu Asn Asp Arg Tyr Ser
Asp Ser Leu Pro Pro Ser Tyr Gly Val Gln Pro Ile
Lys Gly Leu Ala Asp Asn Val Ser Ile Arg Arg Asn
Ser Leu Gly Met Pro Leu Ile Glu Thr Ser Ser Thr
Phe His Asp Ala Leu Phe Thr Leu Gly Tyr Val His
Ala Ser Asp Arg Ile Ser Gln Met Val Gly Leu Arg
Leu Met Ala Gln Gly Arg Leu Ser Glu Met Val Gly
Pro Gly Ala Leu Glu Thr Asp Arg Phe Met Arg Thr
Val Asn Leu Lys Lys Ala Ala Asp Ala Leu Tyr Ala
Gly Ala Ser Pro Arg Leu Lys Arg Phe Phe Glu Thr
Tyr Ala Arg Gly Val Asn Ala Tyr Leu Tyr Arg Tyr
Arg Asp Lys Leu Pro Met Asp Leu Ala Glu Ser Gly
Tyr Arg Pro Glu Tyr Trp Lys Pro Glu Asp Ser Ala
Leu Val Phe Ser Leu Leu Asn Phe Gly Leu Ala Val
Asn Leu Gln Glu Glu Ile Ala Ser Leu Val Leu Ala
Gln Lys Val Gly Ala Asp Lys Leu Pro Trp Leu Thr
Pro Ile Tyr Pro Asp Glu Ala Leu Pro Phe Glu Glu
Ala Asp Lys Leu Lys Gly Leu Arg Leu Asp Gly Arg
Ile Ala Gly Leu Asp Ala Leu Asp Arg Ala Ala Gly
Gln Val Ala Ala Leu Gln Met Met Gly Val Ala Ala
Ser Asn Asn Trp Ala Ile Ala Pro Gln Arg Thr Arg
Ser Gly Lys Ser Ile Leu Ala Asn Asp Thr His Leu
Pro Leu Ser Met Pro Ser Val Trp Asn Tyr Val Gln
Ile Arg Ser Pro Lys Tyr Asn Ala Ala Gly Val Ser
Ile Ala Gly Val Pro Gly Val Val Ala Gly Phe Asn
Gly Lys Leu Ala Trp Gly Met Thr Met Val Met Gly
Asp Asn Gln Asp Leu Phe Leu Glu Gln Val Lys Pro
Gln Gly Gly Lys Leu Tyr Tyr Leu Ala Asp Gly Gln
Trp Lys Pro Ala Ile Glu Arg Asn Glu Thr Phe Phe
Ile Lys Gly Gln Ser Pro Val Arg Glu Val Ile Tyr
Glu Thr Arg His Gly Pro Leu Leu Asn Ser Ala Leu
Gly Glu Arg Lys His Asp Leu Gln Pro Leu Pro Leu
Ser Ser Gly Tyr Gly Ile Ala Tyr Gln Ser Ile Gln
Gly Glu Thr Asp Arg Thr Leu Asp Ala Phe Phe Asp
Leu Ser Arg Ala Lys Asn Val Glu Gln Ala Phe Asp
Ala Thr Arg Asp Val Arg Ala Met Ala Leu Asn Ile
Val Phe Ala Asp Glu Lys His Ile Gly Trp Gln Val
Thr Gly Arg Phe Pro Asn Arg Lys Glu Gly Arg Gly
Leu Leu Pro Ser Pro Gly Trp Asp Gly Arg Tyr Asp
Trp Asp Gly Tyr Ala Asp Pro Ile Leu His Pro Ser
Asp Gln Asp Pro Gln Gln Gly Trp Leu Gly Thr Ala
Asn His Arg Ser Val Gln Pro Gly Tyr Gly Ala Gln
Leu Ser Ser Ser Trp Tyr Tyr Pro Glu Arg Tyr Glu
Arg Ile Ala Gln Leu Ala Gly Gly Ser Lys Ser His
Asp Tyr Arg Ser Met Ile Ala Met Gln Tyr Asp Gln
Thr Thr Pro Phe Ala Gly Lys Leu Gln Ala Met Phe
Asp Ala Pro Gly Met Ala Gln Pro Leu Lys Lys Ala
Ile Asp Ala Leu Pro Ala Asp Gln Arg Ala Arg Ala
Arg Glu Ala Leu Asp Arg Ile Met Ala Phe Asp Gly
Lys Leu Ser Ala Thr Ser Gly Asp Ala Ala Leu Tyr
Glu Ala Phe Leu Gln Glu Ser Ser Lys Gln Ile Phe
Leu Asp Glu Leu Gly Pro Glu Asp Ser Pro Ser Trp
Lys Ala Phe Val Glu Thr Ala Asn Leu Ser Tyr Ser
Ala Gln Ala Asp His Leu Leu Gly Arg Asp Asp Ser
Pro Phe Trp Asp Asp Thr Arg Thr Pro Gln Lys Glu
Asp Lys Pro Thr Ile Leu Ala Arg Ser Leu Ala Ala
Ala Thr Ala Phe Cys Glu Gln Lys Leu Gly Ser Asp
Arg Arg Thr Trp Gln Trp Gly Lys Leu His Thr Tyr
Glu Trp Val Ser Asp Ser Thr Lys Met Ala Pro Asn
Leu Ser Ala Gly Glu Arg Ala Ser Leu Gly Ala Ile
Lys Gly Tyr Leu Asp Arg Gly Pro Tyr Pro Ala Gly
Gly Asp His Ser Thr Leu Asn Val Ser Ala Tyr His
Trp Gly Gln Asp Phe Asn Thr Trp Leu Ile Pro Ala
Met Arg Ile Val Val Asp Phe Gly Gln Ser Glu Pro
Met Ile Gly Leu Asn Ser Ser Gly Gln Ser Gly Asn
Pro Ala Ser Pro His Tyr Ala Asp Gly Ile Asp Ala
Trp Leu Lys Gly Asn Tyr Met Ser Phe Pro Phe Gln
Ser Gln Asn Leu Glu Lys Val Tyr Gly Val Lys Arg
Leu Thr Leu Val Pro Glu Lys.

The present disclosure also provides a method for preparing the above-mentioned N-acyl homoserine lactone acyl transferase, comprising steps of fermenting and culturing a recombinant bacterium expressing N-acyl homoserine lactone acyl transferase in a protein prokaryotic expression vector pET32a, and disrupting the fermented and cultured recombinant bacterium strain, and the performing isolation and purification to give a His-tagged N-acyl homoserine lactone acyl transferase protein.

The present disclosure also provides the use of the recombinant bacterium or the N-acyl homoserine lactone acyl transferase in degrading AHL signaling molecules.

In the present disclosure, the AHL signaling molecules comprises at least one of C4-HSL, C6-SHL, 3—O—C6-HSL, C8-HSL, 3—OH—C8-HSL, C10-HSL, 3—OH—C10—HSL, C12-HSL, 3—O—C12-HSL, 3—OH—C12-HSL, and 3—OH—C14-HSL, and the N-acyl homoserine lactone acyl transferase has a broad-spectrum quenching activity of for different AHL signaling molecules, different substrates and reactivity of N-acyl homoserine lactone acyl transferase may vary.

The present disclosure also provides the use of the gene encoding N-acyl homoserine lactone acyl transferase, or the recombinant vector, or the recombinant bacterium, or N-acyl homoserine lactone acyl transferase in prevention or treating AHL signaling molecule-dependent pathogens.

In the present disclosure, the AHL signaling molecule-dependent pathogen is selected from the grouping consisting of Pectobacterium carotovorum subsp. carotovorum, Burkholderia cepacia, Pseudomonas aeruginosa, Ralstonia solanacearum, Agrobacterium tumefaciens, Dickeya zeae, Erwinia or Pantoea stewartii subsp. stewartii.

In one preferred embodiment of the present disclosure, when the gene encoding N-acyl homoserine lactone acyl transferase encoding gene is used, the gene encoding N-acyl homoserine lactone acyl transferase is expressed in AHL-mediated pathogens, and the recombinant pathogens that successfully express N-acyl homoserine lactone acyl transferase is screened.

In another preferred embodiment of the present disclosure, when the recombinant vector is used, the recombinant vector is introduced into AHL-mediated pathogens, and the recombinant pathogens that successfully express N-acyl homoserine lactone acyl transferase are screened.

In another preferred embodiment of the present disclosure, when the recombinant vector is used, the recombinant vector is introduced into AHL-mediated pathogens, and the recombinant pathogens that successfully express N-acyl homoserine lactone acyl transferase are screened.

This process of screening of the recombinant pathogens that successfully express N-acyl homoserine lactone acyl transferase significantly weakened the production and pathogenicity of the virulence factor of the pathogens.

In the present disclosure, the pathogen is C8-HSL-dependent pathogenic Burkholderia cenocepacia H111, and C4-HSL and 3—OH—C12-HSL-dependent pathogenic Pseudomonas aeruginosa (Pseudomonas aeruginosa) PAO1.

In another preferred embodiment of the present disclosure, when the recombinant vector is used, the recombinant vector is introduced into AHL-mediated pathogens, and the recombinant pathogens that successfully express N-acyl homoserine lactone acyl transferase can:

reduce production of the AHLs; and/or,

reduce mobility; and/or,

reduce formation of biofilm of the pathogens; and/or,

reduce production of protease of the pathogens; and/or,

weaken pathogenicity.

The specific embodiments of the present disclosure will be further described below. It should be noted here that the descriptions of these embodiments are used to help the understanding of the present disclosure, but do not constitute a limitation of the present disclosure. In addition, the technical features involved in the various embodiments of the present disclosure described below can be combined with each other as long as they do not conflict with each other.

The test methods used in the following experimental examples are conventional methods unless otherwise specified, and the materials, reagents, etc. used are commercially available reagents and materials unless otherwise specified.

MM inorganic salt medium: K₂HPO₄, 10.5 g/L; KH₂PO₄, 4.5 g/L; (NH₄)₂SO₄, 2.0 g/L; MgSO₄·7H₂O, 0.2 g/L; FeSO₄, 0.005 g/L; CaCl₂), 0.01 g/L; MnCl₂, 0.002 g/L; glycerol, 2.0 g/L; mannitol, 2.0 g/L; pH 6.8-7.2, sterilized at 121° C. for 20 min.

LB medium: trypton 10.0 g/L, yeast extract 5.0 g/L, NaCl 10.0 g/L, pH 6.8-7.2, sterilization at 121° C. for 15-25 min.

The reagents required in PBS phosphate buffer, X-gal and the culture medium were purchased from biological reagent companies such as Guangzhou Qixiang Biotech Company, Guangzhou Dingguo Biotech Company and etc, and the AHLs used to detect degradation activity in the present disclosure were purchased from Shanghai UDChem Technology Co., Ltd. and Sigma-Aldrich.

Pseudomonas nitroreducens HS-18 was isolated from long-term oil-contaminated soil samples near South China Agricultural University in Guangzhou, and was deposited in the China Center for Type Culture Collection on May 12, 2017 with a deposit number of CCTCC NO: M2017257. HS-18 was cultured at 30° C. using an LB culture medium.

### Example 1 Setting of System for Degradation of AHLs by Pseudomonas Nitroreducens HS-18

First, the strain HS-18 was cultured in LB liquid culture medium in a thermostatic shaker overnight at 30° C., 200 rpm to obtain seed liquid of GD 600=0.5. Three-hundred microliter of seed liquid was taken and fresh LB liquid culture medium of equal volume was added to the seed liquid, and then MOPS (3-(N-morpholino) propanesulfonic acid) having a final concentration of 50 MM, and AHLs with different carbon chain lengths and substituents, having final concentrations of 10-50 iM were added to prepare degradation systems (for different AHLs, the appropriate concentrations were selected based on different color development intensities of the reporter strains), and the degradation systems were cultured in a thermostatic shaker for 12 h, 24 h, and 36 h at 30° C., 200 rpm, and a group without the addition of bacterial suspension was used as a control. AHL-signal molecules that were added externally were extracted using ethyl acetate of the same volume with the degradation system.

TABLE 1
AHLs used to detect the quenching effect of Pseudomonas
nitroreducens HS-18

| Name | Abbreviation | AHL structure |
|---|---|---|
| N-butanoyl-L-homoserine lactone | C4-HSL, BHL | 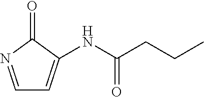 |
| N-hexanoyl-L-homoserine lactone | C6-HSL, HHL | 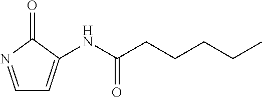 |
| N-(3-oxohexanoyl)-L- homoserine lactone | 3-O-C6-HSL, OHHL | 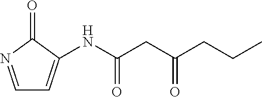 |
| N-octanoyl-L-homoserine lactone | C8-HSL, OHL | 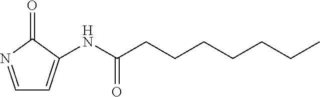 [SMILES: CCCCCCCC(=O)NC1=CC=NC1=O] |
| N-3-hydroxyoctanoyl-L- homoserine lactone | 3-OH-C8-HSL, HOHL | 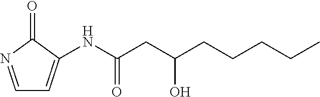 [SMILES: CCCCCC(O)CC(=O)NC1=CC=NC1=O] |
| N-decanoyl-L-homoserine lactone | C10-HSL, DHL | 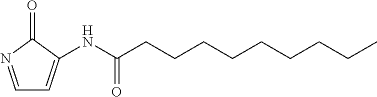 [SMILES: CCCCCCCCCC(=O)NC1=CC=NC1=O] |
| N-3-hydroxydecanoyl-L- homoserine lactone | 3-OH-C10-HSL, HDHL | 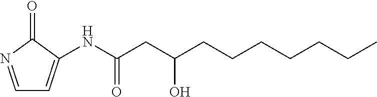 [SMILES: CCCCCCCC(O)CC(=O)NC1=CC=NC1=O] |
| N-dodecanoyl-L-homoserine lactone | C12-HSL, dDHL | 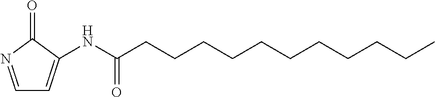 [SMILES: CCCCCCCCCCCC(=O)NC1=CC=NC1=O] |
| N-(3-oxododecanoyl)-L- homoserine lactone | 3-O-C12-HSL, OdDHL | 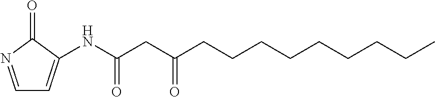 [SMILES: CCCCCCCCCC(=O)CC(=O)NC1=CC=NC1=O] |
| N-(3-hydroxydodecanoyl)-L-homo- serine lactone | 3-OH-C12-HSL, HdDHL | 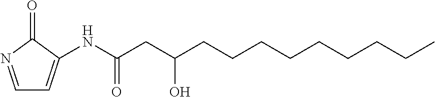 [SMILES: CCCCCCCCCC(O)CC(=O)NC1=CC=NC1=O] |
| N-(3-hydroxytetradecanoyl)-L-ho- moserine lactone | 3-OH-C14-HSL, HtDHL | 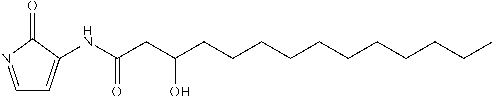 [SMILES: CCCCCCCCCCCC(O)CC(=O)NC1=CC=NC1=O] |

### Example 2 Quantitative Detection of Short-Chain AHLs Using Reporter Strain Chromobacterium Violaceum CV026

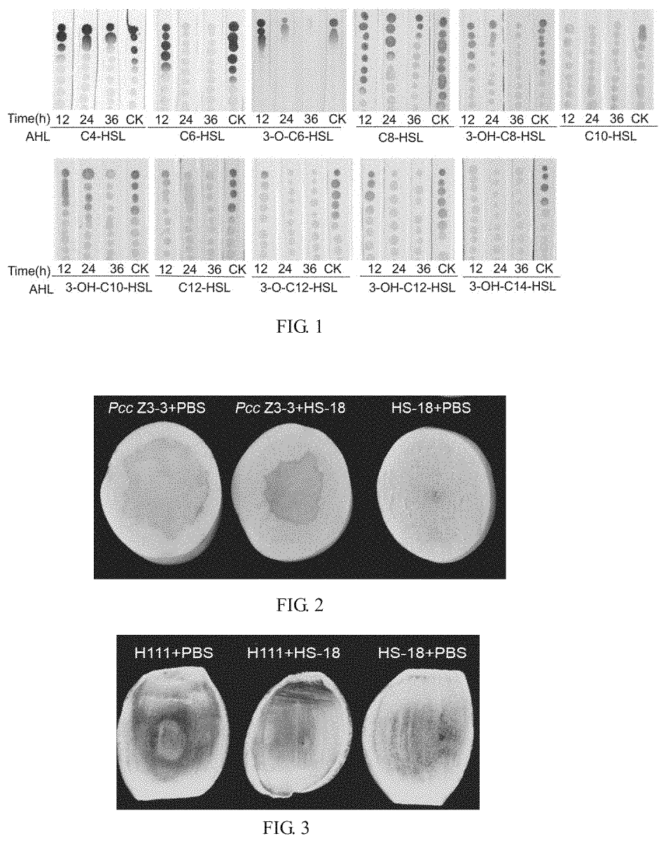

Chromobacterium violaceum CV026 was activated on LB solid plates, and CV026 was cultured in LB liquid medium overnight. The LB solid plates were prepared and cut into 0.8-cm wide agar slice that are spaced with each. 10 μl of the ethyl acetate extract solution from the degradation system as described in Example 1 and spotted the ethyl acetate extract solution on the top of the LB agar slice, and a series of reporter strain droplets with similar sizes were spotted sequentially below the sample loading position. After the droplets of the sample and the reporter strains were dry, the plates were incubated in a thermostatic incubator at 28° C. for 16 h. The area to which the AHLs spread might induce the production of violacein in Chromobacterium violaceum CV026, which made the cells appear purple. The distance at which the purple reporter strain CV026 occurred was proportional to the AHL content to be detected. The results shown in FIG. 1 show that the strain HS-18 of the present disclosure has degradation activity on the short-chain AHLs C4-HSL, C6-HSL, and 3—O—C6-HSL used for detection in the present disclosure.

### Example 3 Quantitative Detection of Medium and Long-Chain AHLs Using Reporter Strain Agrobacterium tumefaciens NT1 (traR, Tra::lacZ749)

Agrobacterium tumefaciens NT1 was activated on LB solid plates, and NT1 was cultured overnight in LB liquid medium containing kanamycin at a final concentration of 50 μg/ml. MM solid plates into which X-gal (5-bromo-4-chloro-3-indole-β-D-galactopyranoside) was added at a final concentration of 40 μg/ml were prepared and cut into 0.8-cm wide Agar slice that were spaced apart with each other. 10p of the ethyl acetate extract solution from the degradation system as described in Example 1 and spotted the ethyl acetate extract solution on the top of the MM agar slice, and a series of reporter strain droplets with similar sizes were spotted sequentially below the sample loading position. After the droplets of the samples and the reporter strains were dry, the plates were wrapped with tin foil and placed still in a thermostatic incubator at 28° C. for 16 h in the dark. The area to which AHLs spread may induce Agrobacterium tumefaciens NT1 to produce β-galactosidase, which decomposes X-gal to make the cells blue. The distance that Agrobacterium tumefaciens NT1 produces blue color was proportional to the AHL content to be detected. The results are shown in FIG. 1, the strain HS-18 of the present disclosure has a degradation activity on the medium- and long-chain AHLs C8-HSL, 3—OH—C8-HSL, C10-HSL, 3—OH—C10-HSL, C12-HSL, 3—O—C12-HSL, 3—OH—C12-HSL, 3—OH—C14-HSL used for detection in the present disclosure.

### Example 4 Study of Biocontrol Effect of Pseudomonas nitroreducens HS-18 on Short-Chain AHL (3—O—C6-HSL)-Dependent Pathogens

In this example, the plant pathogen Pectobacterium carotovorum subsp. carotovora (Pcc) Z3-3 was used as the subject of the biological control research. Pcc-dependent C6-HSL causes soft rot in plants.

The strain HS-18 and the pathogen Pcc Z3-3 were cultured overnight at 30° C. in LB medium, and the seed liquid of HS-18 and Pcc Z3-3 were resuspended in PBS to OD 600=1.0, respectively. The following experimental groups and control groups were set up: 10 μl HS-18+10 μl PBS, 10 μl Pcc Z3-3+10 μl PBS, and 10 μl HS-18+10 μl Pcc Z3-3. Fresh potatoes were used as the host plant of Pcc Z3-3. Before inoculation, the potatoes were washed and cut into about 0.3-cm thick discs. First, HS-18 was inoculated at the center of potato slices, incubated at 30° C. for 1 day, then the pathogen Pcc Z3-3 was inoculated, the slices were cultured at 28° C. for 1 day, and the size of the lesions were observed and measured.

The results shown in FIG. 2 show that the pathogenicity of pathogen Pcc Z3-3 can be significantly weakened after pretreatment of slices of the host plant potato with HS-18 liquid bacterial culture.

### Example 5 Study of Biocontrol Effect of Pseudomonas nitroreducens HS-18 on Medium-Chain AHL (C8-HSL)-Dependent Pathogen

In this example, pathogen Burkholderia cepacia (Bcc) H111 was used as the subject of the biological control research. H111 is C8-HSL-dependent and may cause diseases to plants.

In the present disclosure, LB culture medium was used to culture the strain HS-18 and the pathogen Bcc H111 overnight at 30° C., and HS-18 and Bcc H111 seed liquid were resuspended in PBS to OD 600=1.0, respectively. The following experimental and control groups were set up: 10 μl HS-18+10 μl PBS, 10 μl Bcc H111+10 μl PBS, and 10 μl HS-18+10 μl Bcc H111. Fresh onion was used as host plant of Bcc H111. Before inoculation, the onion was cut into 4 equal parts from the middle of the onion; the onion scales in the middle layer were taken out. First, HS-18 was inoculated at the inner tissue center of the onion scales, incubated at 30° C. for 1 day, and then the pathogen Bcc H111 inoculated and cultured for further 3 days, the lesion size was observed and measured.

The results shown in FIG. 3 show that the disease severity of host plant onion caused by pathogen Bcc H111 can be significantly reduced after continued pretreatment with liquid bacterial culture of HS-18 on the host plant of onion.

### Example 6 Study of Biocontrol Effect of Pseudomonas nitroreducens HS-18 on Short-Chain AHL (3—O—C12-HSL)-Dependent Pathogen

In this example, the pathogen Pseudomonas aeruginosa PAO1 was used as the subject of the biological control research. PAO1 is dependent on C4-HSL and 3—O—C12-HSL and causes plant diseases.

In the present disclosure, strains HS-18 and pathogen PAO1 were cultured in LB culture medium overnight at 37° C., and the seed liquids of HS-18 and PAG1 were separately resuspended in PBS to OD 600=1.0. The following experimental and control groups were set up: 10 μl HS-18+10 μlPBS, 10 μl PAO1+10 μl PBS, and 10 μl HS-18+10 μl PAO1. Fresh lettuce was used as host plant of PAO1. Before inoculation, a piece with a size of about 2 cm*4 cm at the middle position of lettuce stalk was taken out. First, the lettuce stalk tissue was inoculated with HS-18 at the center of lettuce stalk and cultured at 30° C. for 1 day, then inoculated with pathogen PAO1, and the lettuce stalk tissue was continued to culture at 37° C. for 3 days and inoculated at the middle position of lettuce stalk, then the lesion size was observed and counted.

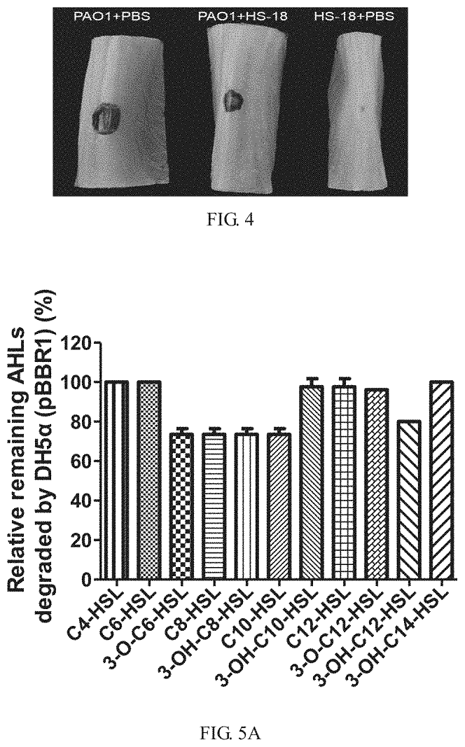

The results in FIG. 4 show that the virulence of pathogen PAO1 to host plant lettuce can be significantly reduced after pretreatment with HS-18.

### Example 7 Acquisition and Identification of the Gene for Degrading AHLs

According to the whole genome sequencing results of Pseudomonas nitroreducens strain HS-18 in previous work, aigC, which may encode N-acyl homoserine lactone acyl transferase, was found using genome annotation and bioinformatics alignment techniques. The software MEGA 5.10 was used to align the amino acid sequences of AigC and the currently known N-acyl homoserine lactone acyl transferase, and ClustalX1.8.3 and the neighbor-joining method were used to analyze the phylogenetic evolution and construct an evolutionary tree. The results shown in FIG. 6 show that AigC has high amino acid similarity (76.6%) with the known AHL acyltransferase HacB PAO1 in Pseudomonas aeruginosa PAO1.

Primers were designed and amplified to construct a broad-host-range vector pBBR1 for insertion of aigC and protein prokaryotic expression vector pET32a. The primer sequences were set as follows:

pBBR1-aigC-F:
gtcgacggtatcgataagcttGCAGAATCGCCGCATAACA,
as set forth in SEQ ID NO: 5;
pBBR1-aigC-R:
cgctctagaactagtggatccTCAGCGACGGACGGGGAT,
as set forth in SEQ ID NO: 6;
pET32a-aigC-F:
gccatggctgatatcggatccATGAAACGCACTTTGACTGTCCT,
as set forth in SEQ ID NO: 7;
pET32a-aigC-R:
ctcgagtgcggccgcaagcttTCAGCGACGGACGGGGAT,
as set forth in SEQ ID NO: 8.

### Example 8 Detection of AHL-Quenching Activity by Recombinant Bacterium DH5a (aigC)

Setting of AHL degradation system: the successfully constructed DH5 α (aigC) was cultured in LB liquid medium overnight to obtain a seed liquid with OD 600=1.0, and fresh LB liquid medium, exogenous AHLs with different carbon chain lengths and substituents at a final concentration of 10-50 μM, and MOPS with a final concentration of 50 mM were added in an equal volume to prepare a degradation system (for different AHL signal molecules, appropriate concentrations are selected based on different intensities of color development of the reporter strain). The degradation system was cultured at 37° C., 200 rpm in a thermostatic shaker. After culturing for 36 h, DH5α (pBBR1) liquid bacterial culture and LB liquid medium without bacterial liquid were used as controls. Next, the liquid culture was extracted with an equal volume of ethyl acetate, and the remaining AHL content in the 10 μl ethyl acetate extract was scaled using the reporter strain.

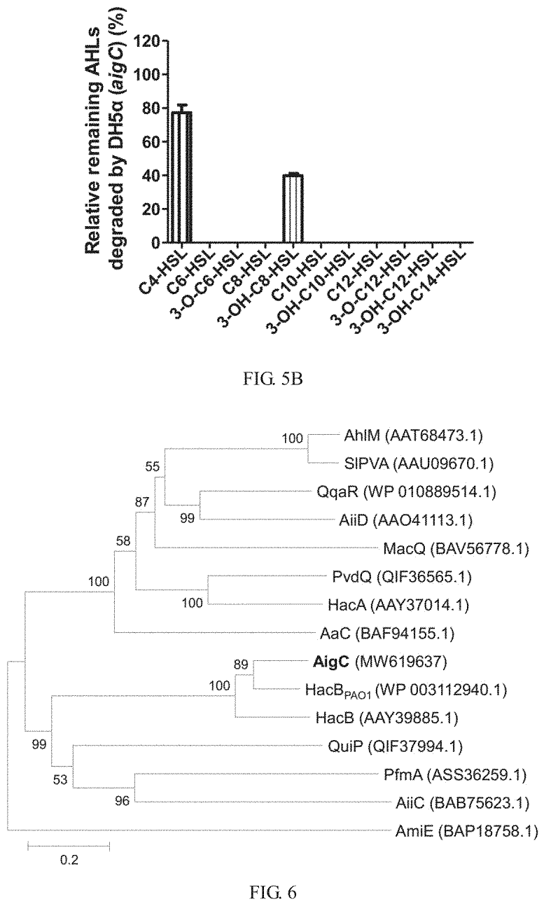

Quantitative detection of short-chain AHLs (C4-C6): Short-chain AHLs (C4-C6) were determined by using the reporter strain Chromobacterium violaceum CV026. Firstly, CV026 was activated on an LB solid plate, and CV026 was cultured overnight at 28° C., 200 rpm in a thermostatic shaker on LB liquid culture medium. An LB solid plate were prepared in a 13 cm*13 cm square petri dish, and cut into 0.8-cm wide agar slice that spaced with each other. 10p of the ethyl acetate extract of the sample to be tested was loaded on the upper end of the agar slice, and droplets of reporter strains of similar size were dotted sequentially below the sample loading position. The area to which the AHLs spread might induce the production of violacein in Chromobacterium violaceum CV026, which made the cells appear purple. After the samples on the agar slice were dry, they were incubated in a thermostatic incubator at 28° C. for 16 hours, and the distance of the purple CV026 bacteria was observed and measured. According to the results in FIG. 5A and FIG. 5B, it is shown that DH5α (aigC) can significantly and efficiently degrade the short-chain AHLs (C4-HSL, C6-HSL, 3—O—C6-HSL) used for detection.

Medium- and long-chain AHLs (C8-C14) were detected using the reporter strain Agrobacterium tumefaciens NT1. First, NT1 was activated on the LB solid plate, and NT1 was cultured overnight at 28° C., 200 rpm in a thermostatic shaker with LB liquid culture containing kanamycin of a final concentration of 50 μg/ml. An MM solid plate was prepared in a 13 cm*13 cm square petri dish and the MM solid plate was cut into 0.8-cm wide agar slice that spaced with each other, and 10 μl of the ethyl acetate extract of the sample to be tested at the upper end of the agar slice was loaded, and the areas below the sample loading place were dotted sequentially a series of droplets of reporter strain in similar size. The area to which AHLs spread can induce Agrobacterium tumefaciens NT1 to produce β-galactosidase, which decomposes X-gal to make the cells appear blue. The distance that Agrobacterium tumefaciens NT1 produces blue color was proportional to the AHL content to be detected. After the samples on the agar slice were dry, they were incubated at 28° C. for 16 hours in a thermostatic incubator, and the distance at which the blue NT1 occurred was observed and measured. According to the results in FIG. 5A and FIG. 5B, it is shown that DH5α (aigC) is very effective in detecting medium and long-chain AHLs (C8-HSL, 3—OH—C8-HSL, C10-HSL, 3—OH—C10-HSL, C12-HSL, 3—O—C12-HSL, 3—OH—C12-HSL, and 3—OH—C14-HSL). Therefore, AHLs with different chain lengths of C4-C14 and different substituents to be tested by aigC have efficient and broad-spectrum quenching activities.

### Example 9 Prokaryotic Expression of AigC Protein

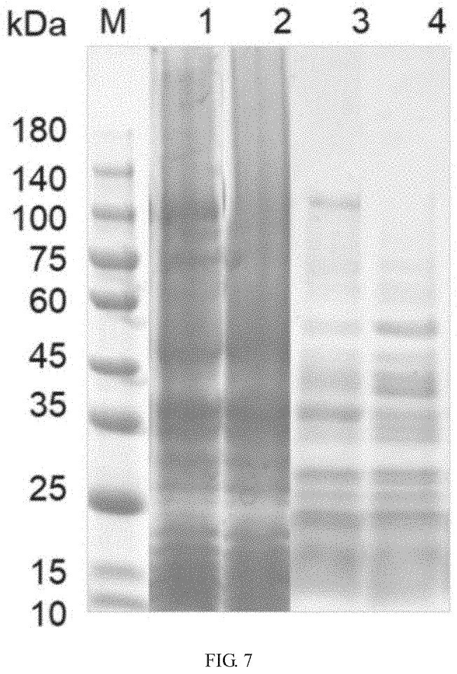

The successfully constructed recombinant bacterium BL21(DE3)(pET32a-aigC) was cultured overnight in LB liquid medium supplemented with a final concentration of 100 μg/ml ampicillin, and cultured at 37° C. and 200 rpm in a thermostatic shaker to obtain a seed liquid. The seed liquid was added to a fresh LB liquid medium containing a final concentration of 100 μg/ml ampicillin in a ratio of 1:100, cultivated to OD 600=0.6-0.8 at 200 rpm in a thermostatic shaker at 37° C., and IPTG of a final concentration of 0.5 mM was added for induction and incubated overnight at 18° C. in a thermostatic shaker at 200 rpm. The cells cultured overnight were collected and disrupted, and the expression of AigC was identified by SDS-PAGE electrophoresis. According to the results in FIG. 7, AigC carrying His-tag can be expressed in normal prokaryotic cells, and the size is about 106.54 kDa.

### Example 10 Effects of aigC Expression on the Growth and on the Intracellular AHL Production of AHL-Mediated Pathogen H111

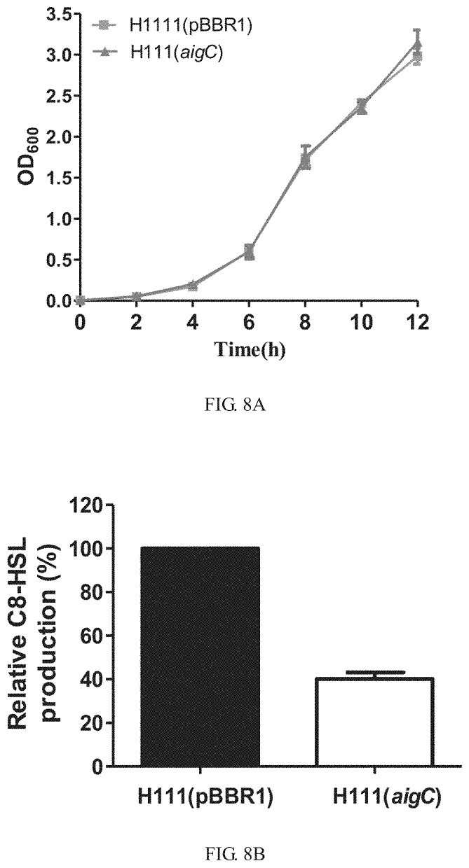

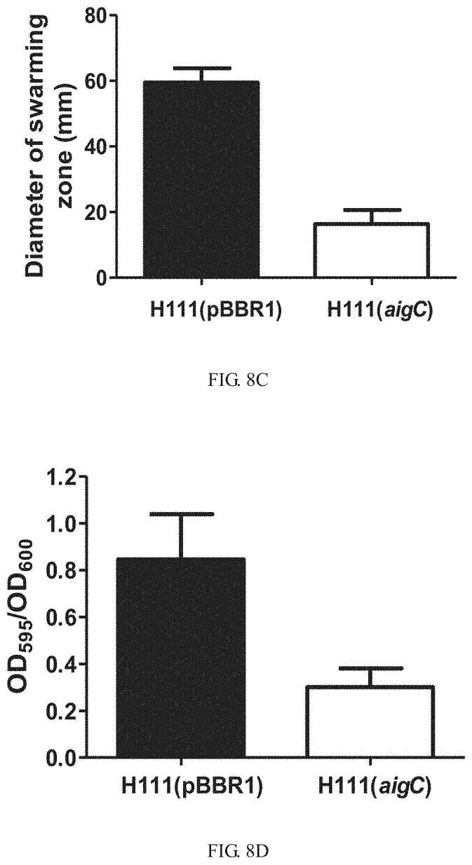

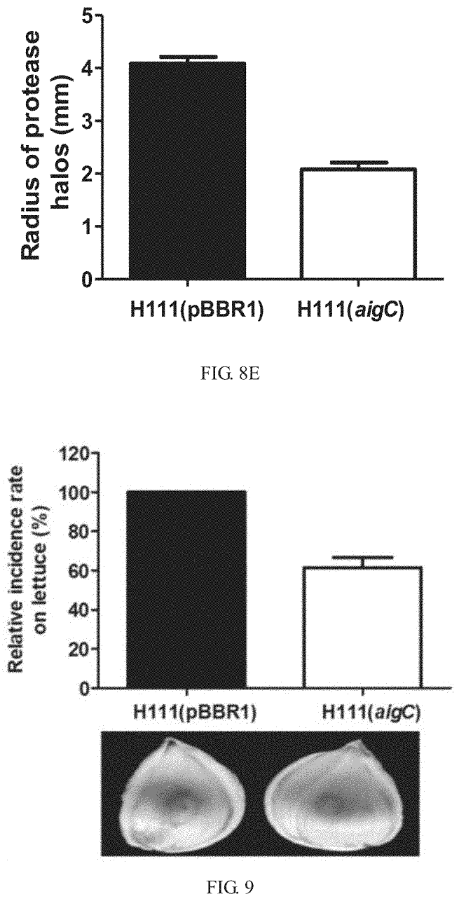

Detection of Growth of H111: the seed liquid of recombinant bacterium H111 (aigC) that had been successfully constructed was cultured overnight to OD 600=0.5, and an LB liquid medium containing the final concentration of 50 μg/ml kanamycin was added in a ratio of 1:100. And the seed liquid was cultured at 30° C., 200 rpm in thermostatic shaker and OD600 was scaled every 2 hours, with H111 (pBBRI) used as a control. The growth curve is shown in FIG. 8A, and the results show that the expression of aigC in H111 does not affect the growth of H111.

The detection of the AHL content produced internally the cell in H111: the seed liquid was cultured overnight to OD 600=0.5, the seed liquid was added in the LB liquid medium containing the final concentration of 50 μg/ml kanamycin in a ratio of 1:100 and cultured at 30° C., 200 rpm for 17 h in thermostatic shaker. An equal volume of ethyl acetate extract of the liquid bacterial culture was taken and the organic phase of the ethyl acetate extract was removed to dryness by rotary evaporation. Then the reporter strain NT1 was added, cultivated for 8 h, the bacteria in the culture liquid was disrupted, and activity of P-galactosidase NT1 production in supernatants after cell disruption was detected. The results are shown as in FIG. 8B, it can be seen at the expression of aigC in H111 significantly reduces the production of C8-HSL in H111.

### Example 11 Effects of aigC Expression on the Growth and on the Intracellular AHL Production of AHL-Mediated Pathogen H111

Detection of motility: a semisolid culture medium (0.8% tryptone, 0.5% glucose, 0.3% agarose) was prepared for the detection of motility of H111. The activated recombinant bacterium H111 (aigC) and control H111 (pBBRI) on the LB plate were dipped with a toothpick and placed in the center of the motility medium plate, and incubated at 30° C. for 17 h in a thermostatic incubator, and the movement diameter was observed and measured. The results are shown in FIG. 8C, it is seen that the expression of aigC significantly weakness the motility of H111.

Biofilm assay: the seed liquid which was cultured overnight was adjusted to OD 600=0.5, and 100 μl of LB liquid medium containing kanamycin with a final concentration of 50 μg/ml in 96-well plate was added in a ratio of 1:100. Biofilm assay was performed at 30° C., 200 rpm for 9 h in a thermostatic shaker, with 8 duplicates for each treatment. First, OD 600 was scaled. Then the liquid bacterial culture was carefully removed with a pipette and discarded. After carefully washing each well with sterile water 3 times, 150 μl of 0.1% crystal violet was added, and it was allowed to stand at room temperature for 15 minutes of staining, and each well was washed with sterile water and air-dried for 3 times. Then 300 μl of 95% ethanol was added, and allowed to stand for 10 min. The absorbance was scaled at a wavelength of 595 nm, and the biofilm formation of the strain was finally calculated by calculating OD 595/GD 600. The results are shown in FIG. 8D. It can be seen that the expression of aigC significantly weakness the formation of biofilm of H111.

Detection of activity of protease: the seed liquid obtained by overnight culture was adjusted to OD 600=0.5, and 20 μl of liquid bacterial culture was added to the well punched with a puncher on the LB+1% skimmed milk plate, with each set having 3 duplicates. The plate was cultured at 30° C. for 17 h in an incubator, and the size of the transparent disks produced by the strain around the well was determined to quantitatively detect the activity of the protease. The results are shown in FIG. 8E, it can be seen that the expression of aigC significantly attenuates the protease production of H111.

### Example 12 Effect of aigC Expression on the Pathogenicity of AHL-Mediated Pathogen H111

The seed liquid of the recombinant bacterium was cultured overnight, and the liquid bacterial culture was resuspended in PBS buffer till OD 600=1.0. Fresh onion was divided into four equal parts and the onion scales were peeled off and used as inoculation tissue. An sterile pipette tip was lightly poked at the wound on the inner center of onion scale, and 20 μl of bacterial suspension was add on wound, with 3 duplicates for each treatment. The onion scales were incubated at 30° C. for 3 days, and the size of the lesions was observed and measured. The results are shown in FIG. 9, it can be seen that the expression of aigC significantly attenuates the pathogenicity of H111.

### Example 13 Effects of aigC Expression on the Growth and on the Intracellular AHL Production of AHL-Mediated Pathogen Pseudomonas aeruginosa PAO1

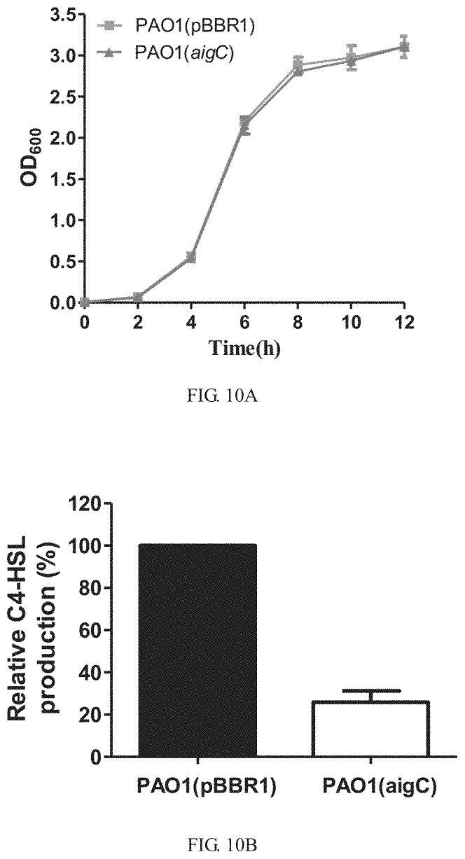

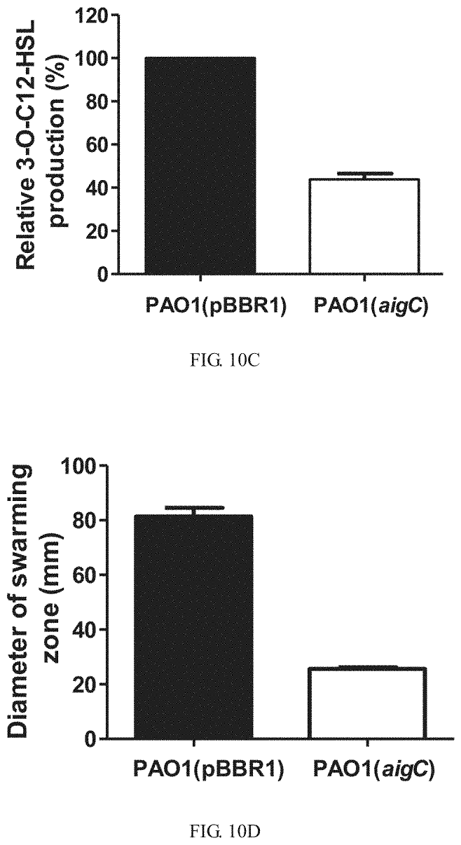

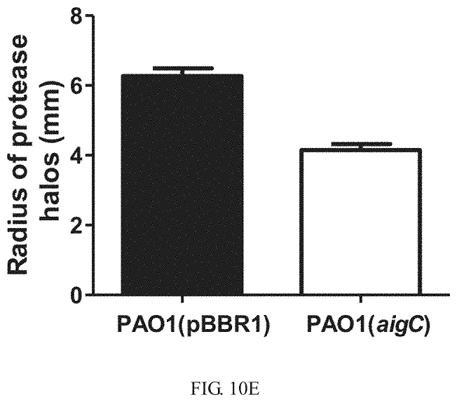

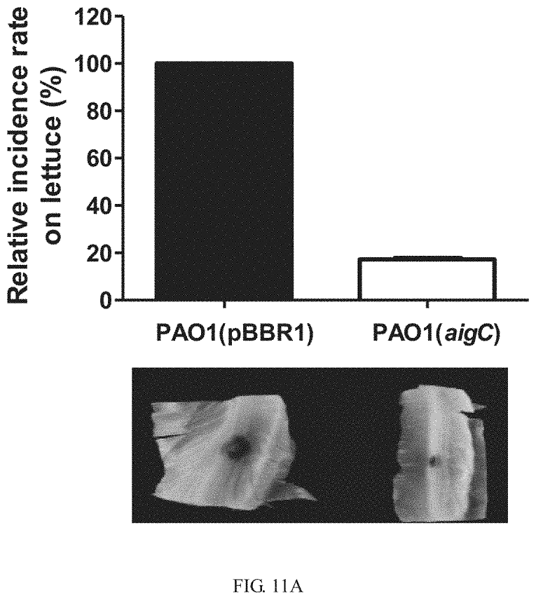

Detection of growth of PAG1: the seed liquid of successfully constructed recombinant bacterium PAG1 (aigC) was cultured overnight to OD 600=0.5, and an LB liquid medium containing the final concentration of 50 μg/ml kanamycin was added in a ratio of 1:100. And the seed liquid was cultured at 37° C., 200 rpm in thermostatic shaker and OD600 was scaled every 2 hours, with PAO1(pBBR1) used as a control. The results of growth curve is shown in FIG. 10A, it can be seen that the expression of aigC in PAG1 does not affect the growth of PAG1.

Determination of AHL content produced intracellularly in PAG1: the seed liquid was cultured overnight to OD 600=0.5, and the resulting seed liquid was added in an LB liquid medium containing final concentration of 50 μg/ml gentamicin in a ratio of 1:100, cultured at 37° C., 200 rpm thermostatic shaker for 17 hours, and an equal volume of ethyl acetate extract of the bacterial liquid was taken. PAO1 can produce two AHLs, namely, C4-HSL and 3—O—C12-HSL.

Detection of the production of C4-HSL: after the ethyl acetate extract was evaporated to dryness, the reporter strain CV026 was added and cultured overnight. The bacterial cells was obtained by centrifugation, the bacterial cells was resuspended in a lysis buffer for lysis of the bacterial cells, and 200 μl of the supernatant after the lysis was taken to detect the absorbance of violacein induced by AHL in CV026 at an absorption wavelength of 545 nm.

Detection of 3—O—C12-HSL production: after the ethyl acetate extract was dried by rotary evaporation, the reporter strain NT1 was added and cultured overnight. Then the bacterial cells were disrupted, and the supernatant after disruption was tested for the activity of the P-galactoside enzyme produced by NT1 induced by AHL. The results are shown in FIG. 10B and FIG. 10C, it can be seen that the expression of aigC in PAO1 significantly reduces the production of C4-HSL and 3—O—C12-HSL in PAO1.

### Example 14 Effect of aigC Expression on the Phenotype of AHL-Mediated Pathogen PAO1

Detection of swarming motility: semisolid culture medium (1% tryptone, 0.5% NaCl, 0.35% agarose) used for detection of PAO1 swarming motility was prepared, and activated recombinant bacterium PAO1 (aigC) and control PAO1 (pBBR1) on the LB plate were dipped with a toothpick dotted in the center of the motility medium plate. The motility medium plate was incubated for static culture at 37° C. for and after 17 hours in athermostatic incubator, the movement diameter was observed and measured. The results are shown in FIG. 10D, it can be seen that the expression of aigC significantly weakens the motility of PAO1.

Detection of activity of the protease: the seed liquid was cultured overnight to OD 600=0.5, and 20 μl of the liquid bacterial culture was taken and added to the wells punched with a puncher on the LB+1% skimmedmilk plate, with 3 duplicates for each well. The plate was cultured at 37° C. for 17 hours in an incubator, and the size of the transparent circle around the well was determined to quantify the activity of the protease. The result are shown in FIG. 10E, it can be seen that the expression of aigC significantly attenuates the protease production of PAO1.

### Example 15 Effects of aigC Expression on the Growth and on the Intracellular AHL Production of AHL-Mediated Pathogen PAO1

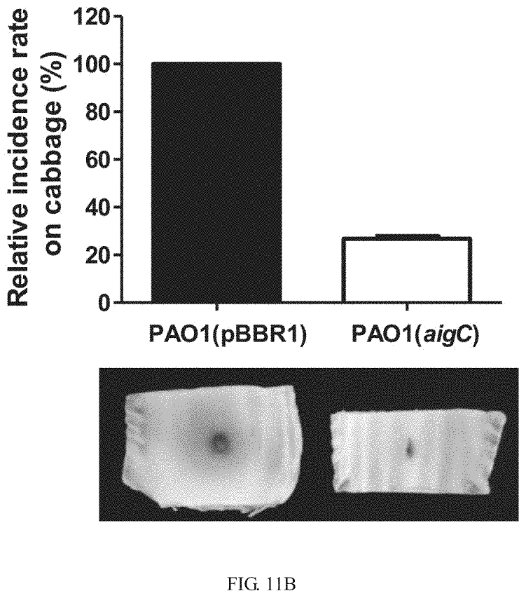

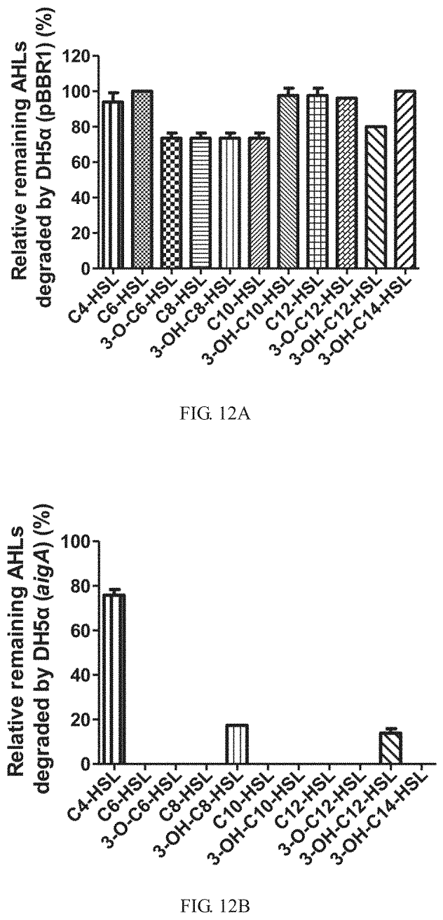

The seed liquid of the recombinant bacterium was cultured overnight, and the liquid bacterial culture was resuspended in PBS buffer till OD 600=1.0. A stalk part with a size of 2 cm*4 cm on fresh lettuce and a stalk part with a size of 5 cm*4 cm on fresh cabbage were lightly poked with a sterile pipette tip at the wound at the center of the surface of the lettuce stalk and cabbage stalk, 20 μl of bacterial suspension was added on the wound, and 3 duplicates were set for each treatment, and the cells were incubated at 30° C. for 3 days, and the size of the lesions was observed and measured. The results are shown in FIG. 11A and FIG. 11B, it can be seen that the expression of aigC significantly weakens the pathogenicity of PAO1.

### Example 16 Acquisition and Identification of Genes Degrading AHLs

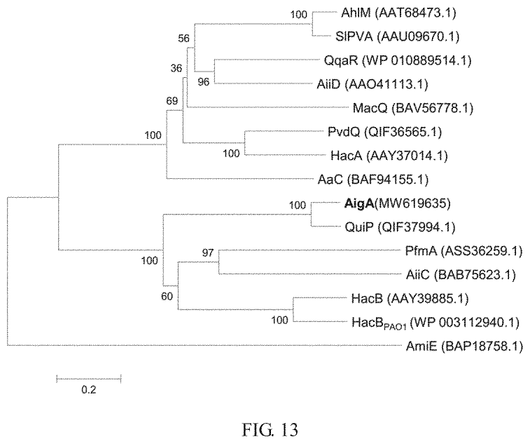

According to the whole genome sequencing results of Pseudomonas nitroreducens strain HS-18 in previous work, genome annotation and bioinformatics alignment techniques were used, a gene, aigA, which may encode N-acyl homoserine lactone acyl transferase, was found. The software MEGA 5.10 was used to align the amino acid sequences of AigA and the currently known N-acyl homoserine lactone acyl transferase, and ClustalX1.8.3 and the neighbor-joining method were used to analyze the phylogenetic evolution and construct an evolutionary tree. The results are shown in FIG. 13, it can be seen that AigA belongs to the AHLs acylase in penicillin G acylase family, and has the highest similarity (82.29%) with the amino acid sequence of QuiP in the known AHL acylase.

The primers were designed and amplified to construct a broad-host-range vector pBBR1 and protein prokaryotic expression vector pET32a recombinant vector for insertion of aigA. The primer sequences are as follows:

pBBR1-aigA-F:
gtcgacggtatcgataagcttTTCCGCGACCCTCTTTGTG,
as set forth in SEQ ID NO: 9;;
pBB R1-aigA-R:
cgctctagaactagtggatccTCACTTCTCCGGCACCAGC,
as set forth in SEQ ID NO: 10;
pET32a-aigA-F:
gccatggctgatatcggatccATGGCCCCGCGTGCCTTC,
as set forth in SEQ ID NO: 11;
pET32a-aigA-R:
ctcgagtgcggccgcaagcttTCACTTCTCCGGCACCAGC,
set forth in SEQ ID NO: 12.

### Example 17 Detection of AHL-Quenching Activity by Recombinant Bacterium DH5α (aigC)

Setting of AHL degradation system: the successfully constructed DH5 α (aigC) was cultured in LB liquid medium overnight to obtain a seed liquid with OD 600=1.0, and fresh LB liquid medium, exogenous AHLs with different carbon chain lengths and substituents at a final concentration of 10-50 μM, and MOPS with a final concentration of 50 mM were added in an equal volume to prepare a degradation system (for different AHL signal molecules, appropriate concentrations are selected based on different intensities of color development of the reporter strain). The degradation system was cultured at 37° C., 200 rpm in a thermostatic shaker. After culturing for 36 h, DH5α (pBBR1) liquid bacterial culture and LB liquid medium without bacterial liquid were used as controls. Next, the liquid culture was extracted with an equal volume of ethyl acetate, and the remaining AHL content in the 10 μl ethyl acetate extract was scaled using the reporter strain.

Quantitative detection of short-chain AHLs (C4-C6): Short-chain AHLs (C4-C6) were determined by using the reporter strain Chromobacterium violaceum CV026. Firstly, CV026 was activated on an LB solid plate, and CV026 was cultured overnight at 28° C., 200 rpm in a thermostatic shaker on LB liquid culture medium. An MM solid plate was prepared and cut into 0.8-cm wide agar slice that spaced with each other, and 10 μl of the ethyl acetate extract of the sample to be tested at the upper end of the agar slice was loaded, and the areas below the sample loading place were dotted sequentially a series of droplets of reporter strain in similar size. The area to which AHLs spread may induce the production of violacein in Chromobacterium violaceum CV026, which makes the cells appear purple. The distance at which the purple reporter strain CV026 occurred was proportional to the AHL content to be detected. After the samples loaded on the agar slice were dry, they were incubated in a thermostatic incubator at 28° C. for 16 hours, and the distance of the purple CV026 bacteria was observed and measured. According to the results shown in FIG. 12A and FIG. 12B, DH5α (aigA) has a significant and efficient AHL-quenching activity on the short-chain AHLs (C4-HSL, C6-HSL, and 3—O—C6-HSL) used for detection.

Medium- and long-chain AHLs (C8-C14) were detected using the reporter strain Agrobacterium tumefaciens NT1. First, NT1 was activated on the LB solid plate, and NT1 was cultured overnight at 28° C., 200 rpm in a thermostatic shaker with LB liquid culture containing kanamycin of a final concentration of 50 μg/ml. An MM solid plate was prepared and cut into 0.8-cm wide agar slice that spaced with each other, and 10 μl of the ethyl acetate extract of the sample to be tested at the upper end of the agar slice was loaded, and the areas below the sample loading place were dotted sequentially a series of droplets of reporter strain in similar size. The area to which AHLs spread, Agrobacterium tumefaciens NT1 can be induced to produce β-galactosidase, which decomposes X-gal to make the cells blue. The distance that Agrobacterium tumefaciens NT1 produces blue color was proportional to the AHL content to be detected. After the samples on the agar slice were dry, they were incubated at 28° C. for 16 hours in a thermostatic incubator, and the distance at which the blue NT1 occurred was observed and measured. The results according to FIG. 12A and FIG. 12B show that DH5α (aigA) has significant and efficient AHL-quenching activity on medium- and long-chain AHLs (C8-HSL, 3—OH—C8-HSL, C10-HSL, 3—OH—C10-HSL, C12-HSL, 3—O—C12-HSL, 3—OH—C12-HSL, and 3—OH—C14-HSL) used for detection.

Therefore, aigA has efficient and broad-spectrum quenching activity on the tested AHLs with different chain lengths of C4-C14 and different substituents.

### Example 18 Prokaryotic Expression of AigC Protein

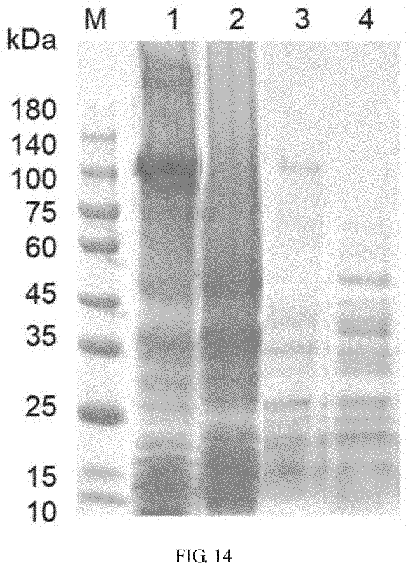

The successfully constructed recombinant bacterium BL21(DE3)(pET32a-aigA) was cultured overnight in LB liquid medium supplemented with ampicillin having final concentration of 100 μg/ml, and cultured at 37° C. and 200 rpm in a thermostatic shaker to obtain a seed liquid. Then the seed liquid was added to a fresh LB liquid medium containing ampicillin having a final concentration of 100 μg/ml in a ratio of 1:100, cultivated to OD 600=0.6-0.8 at 200 rpm in a thermostatic shaker 37° C. Afterwards, IPTG having a final concentration of 0.5 mM was added for induction and incubated overnight at 18° C. 200 rpm in a thermostatic shaker. The cells cultured overnight were collected and disrupted, and the expression of AigA was identified by SDS-PAGE electrophoresis. According to the results in FIG. 14, AigC carrying His-tag can be expressed in normal prokaryotic cells, and the size is about 112.27 kDa.

### Example 19 Effects of aigA Expression on Growth and Intracellular AHL Production of AHL-Mediated Pathogen H111

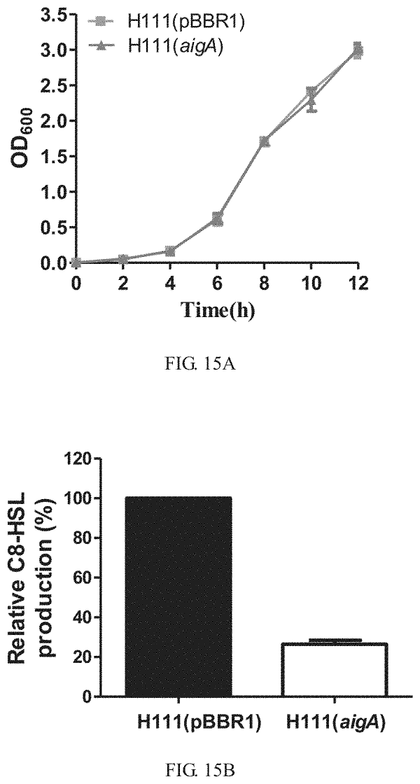

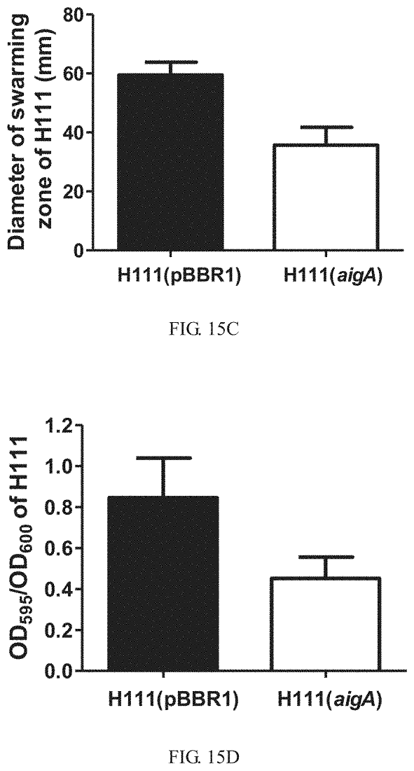

The seed liquid of successfully constructed recombinant bacterium H111 (aigC) was cultured overnight to OD 600=0.5, and an LB liquid medium containing the final concentration of 50 μg/ml kanamycin was added in a ratio of 1:100. And the seed liquid was cultured at 30° C., 200 rpm in thermostatic shaker and OD600 was scaled every 2 hours, with H111 (pBBR1) used as a control. The growth curve is shown in shown in FIG. 15A, and the results show that the expression of aigA in H111 does not affect the growth of H111.

The detection of the AHL content produced internally the cell in H111: the seed liquid was cultured overnight to OD 600=0.5, the seed liquid was added in the LB liquid medium containing the final concentration of 50 μg/ml kanamycin in a ratio of 1:100 and cultured at 30° C., 200 rpm for 17 h in thermostatic shaker. An equal volume of ethyl acetate extract of the liquid bacterial culture was taken and the organic phase of the ethyl acetate extract was removed to dryness by rotary evaporation. Then the reporter strain NT1 was added, cultivated for 8 h, the bacteria in the culture liquid was disrupted, and activity of β-galactosidase NT1 production in supernatants after cell disruption was detected. The results are shown in FIG. 15B, it can be seen that the expression of aigC in H111 significantly reduces the production of C8-HSL in H111.

### Example 20 Effect of aigA Expression on Phenotype of AHL-Mediated Pathogen H111

Detection of motility: a semisolid culture medium (0.8% tryptone, 0.5% glucose, 0.3% agarose) was prepared for the detection of motility of H111. The activated recombinant bacterium H111 (aigC) and control H111 (pBBRI) on the LB plate were dipped with a toothpick and placed in the center of the motility medium plate, and incubated at 30° C. for 17 h in a thermostatic incubator, and the movement diameter was observed and measured. The results are shown in FIG. 15C, it can be seen that the expression of aigA significantly weakened the motility of H111.

Biofilm assay: the seed liquid which was cultured overnight was adjusted to OD 600=0.5, and 100 μl of LB liquid medium containing kanamycin with a final concentration of 50 μg/ml in 96-well plate was added in a ratio of 1:100. Biofilm assay was performed at 30° C., 200 rpm for 9 h in a thermostatic shaker, with 8 duplicates for each treatment. First, OD 600 was scaled, and then the liquid bacterial culture was carefully removed with a pipette and discarded. After carefully washing each well with sterile water 3 times, 150 μl of 0.1% crystal violet was added, and it was allowed to stand at room temperature for 15 minutes of staining, and each well was washed with sterile water and air-dried for 3 times. Then 300 μl of 95% ethanol was added, and allowed to stand for 10 min. The absorbance was scaled at a wavelength of 595 nm, and the biofilm formation of the strain was finally calculated by calculating OD 595/GD 600. The results are shown in FIG. 15D, it can be seen that the expression of aigA significantly weakens the formation of biofilm of H111.

Detection of activity of the protease: the seed liquid was cultured overnight to OD 600=0.5, and 20 μl of the liquid bacterial culture was taken and added to the wells punched with a puncher on the LB+1% skimmed milk plate, with 3 duplicates for each well. The plate was cultured at 30° C. for 17 hours in an incubator, and the size of the transparent circle around the well was determined to quantify the activity of the protease. The result are shown in FIG. 15E, it can be seen that the expression of aigC significantly attenuated the protease production of H111.

### Example 21 Effect of aigA Expression on Pathogenicity of AHL-Mediated Pathogen H111

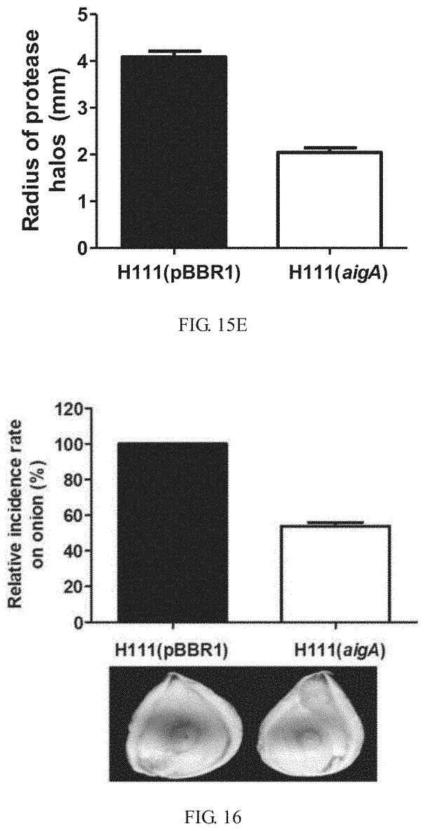

The seed liquid of the recombinant bacterium was cultured overnight, and the liquid bacterial culture was resuspended in PBS buffer till OD 600=1.0. Fresh onion was divided into four equal parts and the onion scales were peeled off and used as inoculation tissue. An sterile pipette tip was lightly poked at the wound on the inner center of onion scale, and 20 μl of bacterial suspension was add on wound, with 3 duplicates for each treatment. The onion scales were incubated at 30° C. for 3 days, and the size of the lesions was observed and measured. The results are shown in FIG. 16, it can be seen that the expression of aigA significantly attenuates the pathogenicity production of H111.

### Example 22 Effect of aigA Expression on the Growth of AHL-Mediated Pathogen Pseudomonas aeruginosa PAO1 and Production of Intracellular AHLs

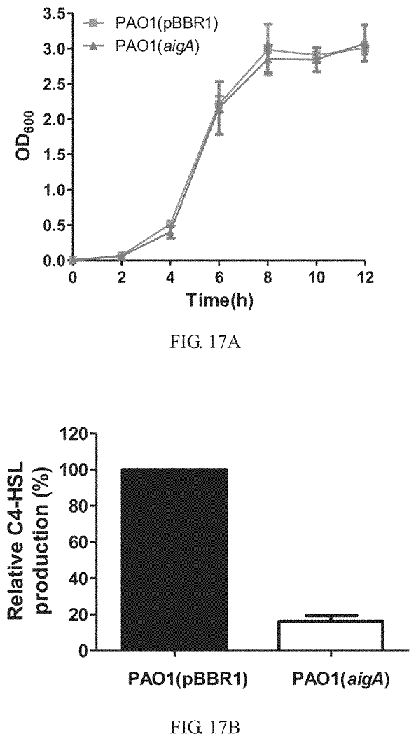

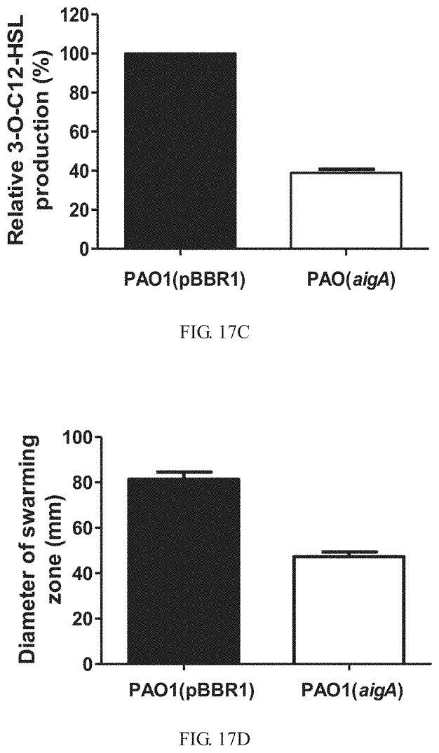

The seed liquid of successfully constructed recombinant bacterium PAO1 (aigA) was cultured overnight to OD 600=0.5, and an LB liquid medium containing the final concentration of 50 μg/ml kanamycin was added in a ratio of 1:100. And the seed liquid was cultured at 37° C., 200 rpm in thermostatic shaker and OD600 was scaled every 2 hours, with the PAO1 (pBBR1) used as a control. The growth curve is shown in FIG. 17A, and the results show that the expression of aigA in PAO1 does not affect the growth of PAG1.

Determination of AHL content produced intracellularly in PAG1: the seed liquid was cultured overnight to OD 600=0.5, and the resulting seed liquid was added in an LB liquid medium containing final concentration of 50 μg/ml gentamicin in a ratio of 1:100, cultured at 37° C., 200 rpm thermostatic shaker for 17 hours, and an equal volume of ethyl acetate extract of the bacterial liquid was taken. PAG1 can generate two AHLs, i.e., C4-HSL and 3—O—C12-HSL.

Detection of the production of C4-HSL: after the ethyl acetate extract was evaporated to dryness, and the reporter strain CV026 was added and cultured overnight. The bacterial cells was obtained by centrifugation, the bacterial cells was resuspended in a lysis buffer for lysis of the bacterial cells, and 200 μl of the supernatant after the lysis was taken to detect the absorbance of violacein induced by AHL in CV026 at an absorption wavelength of 545 nm. Detection of 3—O—C12-HSL production: after the ethyl acetate extract was dried by rotary evaporation, the reporter strain NT1 was added and cultured overnight. Then the bacterial cells were disrupted, and the supernatant after disruption was tested for the activity of the P-galactoside enzyme produced by NT1 induced by AHL. The results is shown in FIG. 17B and FIG. 17C, it can be seen that the expression of aigC in PAG1 significantly reduced the production of C4-HSL and 3—O—C12-HSL in PAO1.

### Example 23 Effect of aigA Expression on Phenotype of AHL-Mediated Pathogen PAO1

Detection of swarming motility: semisolid culture medium (1% tryptone, 0.5% NaCl, 0.35% agarose) used for detection of PAG1 swarming motility was prepared, and activated recombinant bacterium PAG1 (aigC) and control PAG1 (pBBR1) on the LB plate were dipped with a toothpick dotted in the center of the motility medium plate. The motility medium plate was incubated for static culture at 37° C. in a thermostatic incubator, and after 17 hours the movement diameter was observed and measured. The results are shown in FIG. 17D, it can be seen that the expression of aigC significantly weakens the motility of PAG1.

Detection of activity of protease: the seed liquid obtained by overnight culture was adjusted to OD 600=0.5, and 20 μl of liquid bacterial culture was added to the wells punched with a puncher on the LB+1% skimmed milk plate, with 3 duplicates for each set. The plate was cultured at 37° C. for 17 hours in an incubator, and the size of the transparent circle around the well was determined to quantify the activity of the protease. The results shown in FIG. 17E, it can be seen that the expression of aigA significantly attenuates the protease production of PAO1.

### Example 24 Effect of aigA Expression on Pathogenicity of AHL-Mediated Pathogen PAO1

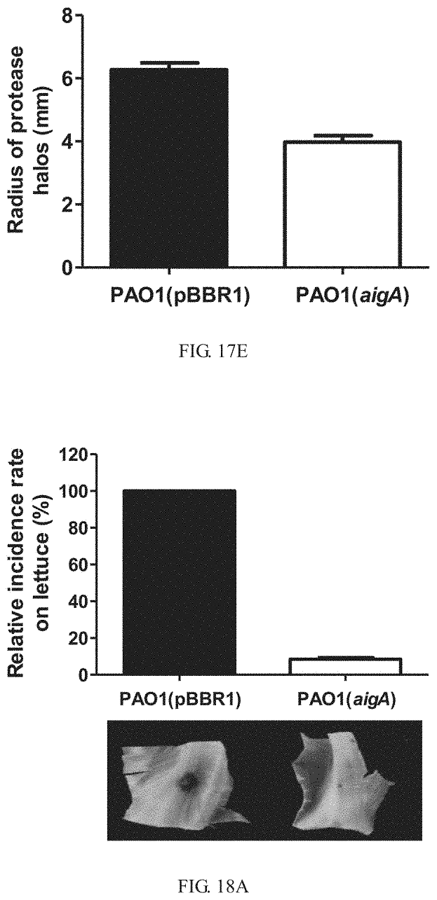

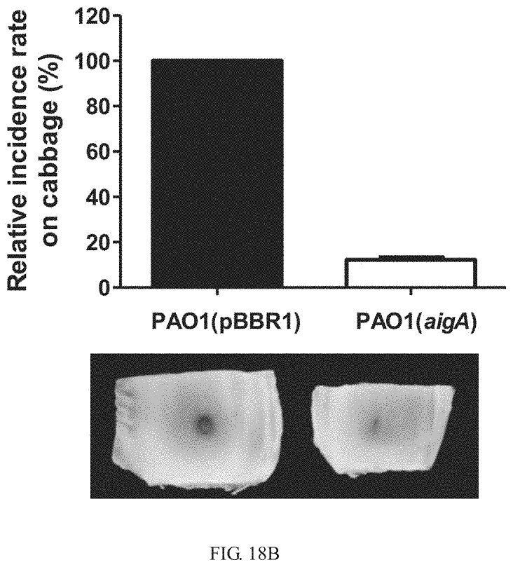

The seed liquid of the recombinant bacterium was cultured overnight, and the liquid bacterial culture was resuspended in PBS buffer till OD 600=1.0. A stalk part with a size of 2 cm*4 cm on fresh lettuce and a stalk part with a size of 5 cm*4 cm on fresh cabbage were lightly poked with a sterile pipette tip at the wound at the center of the surface of the lettuce stalk and cabbage stalk, 20 μl of bacterial suspension was added on the wound, and 3 duplicates were set for each treatment, and the cells were incubated at 30° C. for 3 days, and the size of the lesions was observed and measured. The results are shown in FIG. 18A and FIG. 18B, and it can be seen that the expression of aigA significantly weakened the pathogenicity of PAO1.

Described above are only the preferred embodiments of the present disclosure. It should be pointed out that several improvements and modifications can be made by those skilled in the art without departing from the principles of the present disclosure. These improvements and modifications should be regarded as falling within the protection scope of the present disclosure.

What is claimed is:

1. A method for degrading a AHL signal molecule by using Pseudomonas nitroreducens HS-18, wherein the AHL signal molecule is selected from the group consisting of C4-HSL, C6-SHL, 3—O—C6-HSL, C8-HSL, 3—OH—C8-HSL, C10-HSL, 3—OH—C10—HSL, C12-HSL, 3—O—C12-HSL, 3—OH—C12-HSL, and 3—OH—C14-HSL.

2. The method according to claim 1, wherein pH and temperature for degrading the AHL signaling molecule using pseudomonas nitroreducens HS-18 are 6.8-7.2 and 28-37° C., respectively.

3. A method for preventing and treating AHL signaling molecule-dependent pathogens by using pseudomonas nitroreducens HS-18, comprising a step of co-inoculating a liquid bacterial culture containing Pseudomonas nitroreducens HS-18 and the AHL signaling molecule-dependent pathogens in a plant.

4. The method according to claim 3, wherein co-inoculating is conducted by steps of mixing a liquid bacterial culture containing Pseudomonas nitroreducens HS-18 with the AHL signaling molecule-dependent pathogens, and applying to diseased part of the plant.

5. The method according to claim 3, wherein the AHL signaling molecule-dependent pathogen is selected from the grouping consisting of Pectobacterium carotovorum subsp. carotovorum, Burkholderia cepacia, Pseudomonas aeruginosa, Ralstonia solanacearum, Agrobacterium tumefaciens, Dickeya zeae, Erwinia or Pantoea stewartii subsp. stewartii.

6. The method according to claim 5, wherein the AHL signaling molecule-dependent pathogen is selected from the group consisting of 3—O—C6-HSL-dependent pathogenic Pectobacterium carotovorum subsp. carotovorum, C8-HSL-dependent pathogenic Burkholderia cepacia, C4-HSL-dependent and 3—OH—C12-HSL-dependent pathogenic Pseudomonas aeruginosa, C6-HSL-dependent and C8-HSL-dependent pathogenic Ralstonia solanacearum, 3—O—C8-HSL-dependent pathogenic Agrobacterium tumefaciens, 3—O—C6-HSL-dependent pathogenic Dickeya zeae, 3—O—C6-HSL-dependent pathogenic Erwinia.

7. The method according to claim 3, wherein the preventing and treating AHL signaling molecule-dependent pathogen comprises performing one or more of the following steps:
(1) reducing production of the AHLs; and/or,
(2) reducing motility of the pathogen; and/or,
(3)reducing formation of biofilm of the pathogen; and/or,
(4) reducing production of protease of the pathogen; and/or,
(5) weakening pathogenicity of the pathogen.

8. A gene encoding N-acyl homoserine lactone acyl transferase derived from the Pseudomonas nitroreducens HS-18, comprising a gene aigA or a gene of aigC; wherein the nucleotide sequence of the gene aigA is set forth in SEQ ID NO: 1, and the nucleotide sequence of the gene aigC is set forth in SEQ ID NO: 2.

9. A recombinant vector, comprising the gene encoding N-acyl homoserine lactone acyl transferase of claim 8, which is inserted into the recombinant vector.

10. The recombinant vector according to claim 9, wherein the recombinant vector is a recombinant vector obtained by inserting the gene aigA or the gene aigC into the broad-host vector pBBR1 for heterologous expression of the gene encoding N-acyl homoserine lactone acyl transferase; or,
the recombinant vector is a recombinant vector for protein prokaryotic expression of N-acyl homoserine lactone acyl transferase and is obtained by inserting the gene aigA or the gene aigC into a protein prokaryotic expression vector pET32a.

10. A recombinant bacterium comprising the recombinant vector of claim 8 or claim 9.

11. The recombinant bacterium according to claim 10, wherein the recombinant bacterium is a non-pathogenic bacteria or an AHL signaling molecule-dependent pathogen.

12. A formulation for preventing and treating AHL signaling molecule-dependent pathogen or for degrading an AHL signaling molecule, wherein the formulation comprises the recombinant bacterium of claim 10 or claim 11, or comprises N-acyl homoserine lactone acyl transferase; and amino acid sequence of N-acyl homoserine lactone acyl transferase is set forth in SEQ ID NO: 3 or SEQ ID NO: 4.


SEQUENCE LISTING (12 sequences)

SEQ ID NO: 1 - DNA, 2541 bp, Artificial Sequence
  nucleotide sequence of the gene aigA
atggccccgcgtgccttccgccggcttcccagcttcggccttgccgccaccgtcactgccgctgtcagccttaccggctgccagggctggctgaatgaccgctattccgacagcctgccgccgagctacggcgtgcagccgatcaagggcctggccgacaacgtgtcgatccgccgcaacagcctgggcatgccgctgatcgagaccagcaccttccacgacgcgctgttcaccctcggttatgtgcacgccagcgaccgcatcagccagatggtcggcctgcgcctgatggcccagggtcgcctgtcggaaatggtcggccccggcgccctggaaaccgaccgattcatgcgcacggtgaacctgaagaaggccgcggacgccctctacgccggcgcctcaccgcgcctcaagcgtttcttcgagacctacgcgcgcggcgtcaacgcctacctgtaccgctaccgcgacaagctgccgatggacctcgccgagtccggctaccgccccgagtactggaagccggaagattccgcactggtcttcagcctgctgaacttcggcctggcggtgaacctgcaggaggaaatcgcctccctggtactggcgcagaaagtcggtgccgacaagctgccctggctgacgccgatctatccggacgaagcgctgccgttcgaggaagcggacaaactcaagggcctgcgcctggacggcaggatcgccggcctcgacgccctggatcgtgcggcaggccaggtcgccgccctgcagatgatgggcgtcgccgcctccaacaactgggccatcgccccgcagcgcacgcgcagcggcaagagcatcctggccaacgacacccacctgccgctgtccatgccctcggtgtggaactacgtgcagatccgctcgccgaaatacaacgccgcgggcgtctccatcgccggcgtaccgggcgtggtcgccggtttcaacggcaagctggcctggggcatgaccatggtcatgggcgacaaccaggaccttttccttgagcaggtgaagccccagggcggcaagctctactacctcgccgatggccagtggaaaccggccatcgagcgcaatgaaaccttcttcatcaagggccagagcccggtccgcgaagtcatctacgagactcgccacggcccgctgctcaacagcgcgctgggcgagcgcaagcacgacctgcaaccgctgccgctgtccagcggctacggcatcgcctaccagagcatccagggcgagaccgaccgcaccctcgacgccttcttcgacctgtcccgcgcgaagaacgtcgagcaggccttcgacgccacccgcgacgtgcgcgccatggcgctgaacatcgtgttcgccgacgagaagcacatcggctggcaggtcaccgggcgcttccccaaccgcaaggaaggccgcggcctgctgccttcgcccggctgggacggccgctatgactgggacggctacgccgatccgatcctccacccgtccgaccaggacccgcagcagggctggctgggcaccgccaaccaccgcagcgtgcagccaggctacggcgcgcagctgtccagttcctggtactacccggagcgctacgagcgcatcgcccaactcgccggcggcagcaagagccacgactaccgcagcatgatcgccatgcagtacgaccagaccacgccctttgccggcaagctgcaggccatgttcgatgcaccgggcatggcgcagccgctgaagaaagccatcgacgccctgcccgccgaccagcgcgcccgcgcccgcgaagcgctggaccggatcatggcgttcgacggcaagctctcggcgacctccggcgacgccgccctgtacgaagccttcctgcaggagagcagcaagcagatcttcctcgatgaactgggcccggaggacagcccgtcctggaaagccttcgtcgagaccgccaacctctcctactcggcccaggccgaccacctgctgggccgtgacgacagcccgttctgggatgacacccgcaccccgcagaaggaagacaaaccgaccatcctcgcccgcagcctggccgccgccaccgccttctgcgaacagaagctgggcagcgaccgccgtacctggcagtggggcaagctgcacacctacgagtgggtcagcgacagcaccaagatggcgcccaacctcagtgccggcgagcgcgccagcctcggcgcgatcaagggctacctggaccgcggcccctacccggccggcggcgaccacagcacgctgaacgtctcggcctaccactggggccaggacttcaacacctggctgatcccggccatgcgcatcgtcgtcgacttcggccagagcgaaccgatgatcggccttaacagcagtggccagtccggcaacccggccagcccgcactacgcagacggcatcgacgcctggctgaagggcaactacatgagcttccccttccagtcgcagaaccttgagaaggtctacggcgtgaagcgcctgacgctggtgccggagaagtga

SEQ ID NO: 2 - DNA, 2388 bp, Artificial Sequence
  nucleotide sequence of the gene aigC
atgaaacgcactttgactgtcctcgctgtcgttgtcgtcgctgccgcggcgggcgctggctggtacctgcacggcaagcagccggtacgcgacggacaactgccgctggccggcctcgccagcgaagtcacggttcgctacgacgagcgtggcgtgccgcacatcaaggccggcagcgaggaggacatgtaccgggcgatcggttacgtgcatgcccaggaccggctgttccagatggaaatactgcggcgcctgtcccgcggcgagctggccgaagtgctcgggcccaagctggtggacaccgaccgcatgttccgtagcctgcgcatccgcgaccacgcggcggagtatgtgaagacgcaggacaagaattccccggcctggaaggccctggtcgcctacctcgatggcgtcaaccagttccaggacagccacccgcgcccggtggagttcgacatcctcggcatacccaagcgccccttcacgcccgaggacaccgtcagcgtcgccggctacatggcctacagcttcgccgccgccttccgtaccgaaccggtgctgacctacgtgcgcgaccaactgggcgcggattacctgaaagtcttcgacctcgactggcaccccaacggcgtcctcacgccgtccccgttggccgccgccgactggcaggacatgagcgccatcgcccagctcagccacgcggccctggagaaggccggcctgccgcagttcgaaggcagcaacgcctgggccgtgtccggcagccgcaccaagagcggcaagccgctgctggcgggtgacccacacatccgcttcgccgtgccggcagtgtggtacgagatgcaggccagcgcgccggggttcgagctgtacggccactaccaggcgctcaacccgttcgcctcgctgggccataacctgcagttcggctggagcctgaccatgttccagaacgacgacgtcgacctggtcgccgagaaggtcaacccggacaaccccaaccaggtctggtaccacggccagtgggtggacctgaagagcgaggagcagagcatcgccgtgaaaggcgaggcgccagtgaagatcaccctgcgcagctcgccccacggtccgctggtcaatgacgcgctgggcactgccgcgggcaagacaccggtggccatgtggtgggccttcctggaaacccagaacccgatcctcgatgccttctacgagctgaaccgcgccgatacccttgccaaggcccgcacggcggcttcgaagatccagtcgccgggcctcaacgtggtatgggccaacgcccgcggcgacatcggctggtgggcatcggcgcaattgccggtacgcccggacggggtcaacccgaacttcctgctcgacggcgccagcggccaggccgacaagaccggcttctaccccttcagcgagaacccgcaggaagaaaacccggcgcgcggctacatcgtctcggccaacttccagccggtaccggccaacggtcgcccggtgccgggctactacaacctgcccgaccgcggtcagcaactgaacaagcgcctgtccgacgattcggtgaaatgggacctgcagaacagccaggcgctgcaactggacaccgccaccggctatggcccgcgcttcctcaagccgctgctgccgatcctgcgtgaagccgcggcaaccgacgaagagaaggcgctggtggagagcctggccaactggcagggcgaccatccgctggactccgtgaccgccacgctgttcaaccagctcctctaccaggtggcagacggcgccatgcgcgacgagatgggcgacgccttcttcgacaacctgctgtccacccgcgtgctcgacgtggccctgccgcgcctggccgccgacgagggctcgccctggtgggacaaccgcaagacgccgcagaaggaaactcgcgcggatatcgtcaaggccgcctggaaaggcagcctggcgcatctgcgcaccaccctcggccaggactcgaaacagtggctgtggggtaaggcgcacaccctgacccacggccacccgctgggccagcagaagccgctggatcgcctcttcaatgtcggcccattcgccgcgccgggcggccacgaggtaccgaacaacctctccgcccgcgtcggcccggcaccctggcaggtggtctacggcccgtccacccgtcgcctgatcgacttcgccgacccggcccacagcctgggcatcaacccggtgggccagagcggcgtgcccttcgacaagcactacgacgaccaggccgaagcctacatcgaaggccagtacctgccgcagcactacgatgaaaacgaggtgaaggccaacaccaagggcatcctgcggctgatccccgtccgtcgctga

SEQ ID NO: 3 - protein, 795 aa, Artificial Sequence
  amino acid sequence of N-acyl homoserine lactone acyl transferase
MKRTLTVLAVVVVAAAAGAGWYLHGKQPVRDGQLPLAGLASEVTVRYDERGVPHIKAGSEEDMYRAIGYVHAQDRLFQMEILRRLSRGELAEVLGPKLVDTDRMFRSLRIRDHAAEYVKTQDKNSPAWKALVAYLDGVNQFQDSHPRPVEFDILGIPKRPFTPEDTVSVAGYMAYSFAAAFRTEPVLTYVRDQLGADYLKVFDLDWHPNGVLTPSPLAAADWQDMSAIAQLSHAALEKAGLPQFEGSNAWAVSGSRTKSGKPLLAGDPHIRFAVPAVWYEMQASAPGFELYGHYQALNPFASLGHNLQFGWSLTMFQNDDVDLVAEKVNPDNPNQVWYHGQWVDLKSEEQSIAVKGEAPVKITLRSSPHGPLVNDALGTAAGKTPVAMWWAFLETQNPILDAFYELNRADTLAKARTAASKIQSPGLNVVWANARGDIGWWASAQLPVRPDGVNPNFLLDGASGQADKTGFYPFSENPQEENPARGYIVSANFQPVPANGRPVPGYYNLPDRGQQLNKRLSDDSVKWDLQNSQALQLDTATGYGPRFLKPLLPILREAAATDEEKALVESLANWQGDHPLDSVTATLFNQLLYQVADGAMRDEMGDAFFDNLLSTRVLDVALPRLAADEGSPWWDNRKTPQKETRADIVKAAWKGSLAHLRTTLGQDSKQWLWGKAHTLTHGHPLGQQKPLDRLFNVGPFAAPGGHEVPNNLSARVGPAPWQVVYGPSTRRLIDFADPAHSLGINPVGQSGVPFDKHYDDQAEAYIEGQYLPQHYDENEVKANTKGILRLIPVRR

SEQ ID NO: 4 - protein, 846 aa, Artificial Sequence
  amino acid sequence of N-acyl homoserine lactone acyl transferase
MAPRAFRRLPSFGLAATVTAAVSLTGCQGWLNDRYSDSLPPSYGVQPIKGLADNVSIRRNSLGMPLIETSTFHDALFTLGYVHASDRISQMVGLRLMAQGRLSEMVGPGALETDRFMRTVNLKKAADALYAGASPRLKRFFETYARGVNAYLYRYRDKLPMDLAESGYRPEYWKPEDSALVFSLLNFGLAVNLQEEIASLVLAQKVGADKLPWLTPIYPDEALPFEEADKLKGLRLDGRIAGLDALDRAAGQVAALQMMGVAASNNWAIAPQRTRSGKSILANDTHLPLSMPSVWNYVQIRSPKYNAAGVSIAGVPGVVAGFNGKLAWGMTMVMGDNQDLFLEQVKPQGGKLYYLADGQWKPAIERNETFFIKGQSPVREVIYETRHGPLLNSALGERKHDLQPLPLSSGYGIAYQSIQGETDRTLDAFFDLSRAKNVEQAFDATRDVRAMALNIVFADEKHIGWQVTGRFPNRKEGRGLLPSPGWDGRYDWDGYADPILHPSDQDPQQGWLGTANHRSVQPGYGAQLSSSWYYPERYERIAQLAGGSKSHDYRSMIAMQYDQTTPFAGKLQAMFDAPGMAQPLKKAIDALPADQRARAREALDRIMAFDGKLSATSGDAALYEAFLQESSKQIFLDELGPEDSPSWKAFVETANLSYSAQADHLLGRDDSPFWDDTRTPQKEDKPTILARSLAAATAFCEQKLGSDRRTWQWGKLHTYEWVSDSTKMAPNLSAGERASLGAIKGYLDRGPYPAGGDHSTLNVSAYHWGQDFNTWLIPAMRIVVDFGQSEPMIGLNSSGQSGNPASPHYADGIDAWLKGNYMSFPFQSQNLEKVYGVKRLTLVPEK

SEQ ID NO: 5 - DNA, 40 bp, Artificial Sequence
  sequence of primer pBBR1-aigC-F
gtcgacggtatcgataagcttgcagaatcgccgcataaca

SEQ ID NO: 6 - DNA, 39 bp, Artificial Sequence
  sequence of primer pBBR1-aigC-R
cgctctagaactagtggatcctcagcgacggacggggat

SEQ ID NO: 7 - DNA, 44 bp, Artificial Sequence
  sequence of primer pET32a-aigC-F
gccatggctgatatcggatccatgaaacgcactttgactgtcct

SEQ ID NO: 8 - DNA, 39 bp, Artificial Sequence
  sequence of primer pET32a-aigC-R
ctcgagtgcggccgcaagctttcagcgacggacggggat

SEQ ID NO: 9 - DNA, 40 bp, Artificial Sequence
  sequence of primer pBBR1-aigA-R
gtcgacggtatcgataagcttttccgcgaccctctttgtg

SEQ ID NO: 10 - DNA, 40 bp, Artificial Sequence
  sequence of primer pBBR1-aigA-F
cgctctagaactagtggatcctcacttctccggcaccagc

SEQ ID NO: 11 - DNA, 39 bp, Artificial Sequence
  sequence of primer pET32a-aigA-F
gccatggctgatatcggatccatggccccgcgtgccttc

SEQ ID NO: 12 - DNA, 40 bp, Artificial Sequence
  sequence of primer pET32a-aigA-F
ctcgagtgcggccgcaagctttcacttctccggcaccagc